(19) United States
(12) Patent Application Publication
(10) Pub. No.: US 2025/0382272 A1
(43) Pub. Date: Dec. 18, 2025

(54) CRYSTALLINE FORM OF INDAZIFLAM, METHODS FOR ITS PREPARATION AND USE THEREOF

(71) Applicant: ADAMA AGAN LTD., Ashdod (IL)

(72) Inventors: Einat KISIN-FINFER, Hod Hasharon (IL); Reut FALLEK, Ashdod (IL); Ira AGRANOVICH, Ashdod (IL); Marwan E. SIMAAN, Nazareth (IL); Leonid ERCHOV, Beit Shemesh (IL); Judith ARONHIME, Rehovot (IL)

(73) Assignee: ADAMA AGAN LTD., Ashdod (IL)

(21) Appl. No.: 18/879,164

(22) PCT Filed: Jun. 29, 2023
(86) PCT No.: PCT/IL2023/050679

§ 371 Date: Dec. 26, 2024

(30) Foreign Application Priority Data
Jun. 30, 2022 (EP) 22182294.3
Dec. 29, 2022 (EP) 22217081.3

(51) Int. Cl. C07D 251/18; A01N 43/68; A01P 13/00; C07C 227/08; C07C 279/26

(52) U.S. Cl. CPC C07D 251/18; A01N 43/68; A01P 13/00; C07C 227/08; C07C 279/26

(57) ABSTRACT

A crystalline form of indaziflam, processes for the preparation thereof, an agrochemical composition containing the crystalline form of indaziflam and a process for the preparation of the agrochemical composition.

### FIELD OF THE INVENTION

The present invention belongs to the field of agrochemistry. It is directed to a novel crystalline form of N-[(1R,2S)-2,3-dihydro-2,6-dimethyl-1H-inden-1-yl]-6-(1-fluoroethyl)-1,3,5-triazine-2,4-diamine (Indaziflam.

### BACKGROUND

Triazine compounds are a class of compounds suitable for being used as herbicides. Triazine compounds such as atrazine, ametryne, indaziflam or triaziflam are among the compounds that are used as herbicides.

The prior art discloses in EP1592674 A1, EP2231679 A1 or EP3347342 A1 different methods for preparing indaziflam. However, none of the methods disclose a crystalline form of indaziflam.

Therefore, there is the need to provide a novel crystalline form of indaziflam which has improved properties. It would be advantageous if the crystalline form of indaziflam could provide improved flowability. Flowability is a feature of significant importance to achieve a good manufacturing efficiency and product quality in solid processing industries, to avoid capacity shortfall and production interruption.

### SUMMARY OF THE INVENTION

A first aspect of the invention is a crystalline form of indaziflam which has a compressibility index lower or equal to 17.

A second aspect of the invention is a process for the preparation of a crystalline form of indaziflam which has a compressibility index lower or equal to 17, wherein indaziflam is recrystallized in a solvent selected from the group consisting of alcohols, alcohols mixed with water, amides, lactams, esters of carboxylic acids, optionally halogenated aliphatic or aromatic solvents.

A third aspect of the invention is an agrochemical composition which comprises a crystalline form of indaziflam which has a compressibility index lower or equal to 17.

A fourth aspect of the invention is a process for the preparation of an agrochemical composition comprising indaziflam, wherein the process comprises a step of pulverizing a powder or a slurry comprising a crystalline form of indaziflam which has a compressibility index lower or equal to 17, and a step of mixing the pulverized powder or slurry with one or more agriculturally acceptable inert additives or adjuvants of the agrochemical composition.

### BRIEF DESCRIPTION OF THE DRAWINGS

FIG. 1 is an X-ray powder diffractogram (XRD) of the crystalline form of indaziflam of the present invention from a sample obtained from example 1 and recorded using Cu-Kα radiation at 25° C.

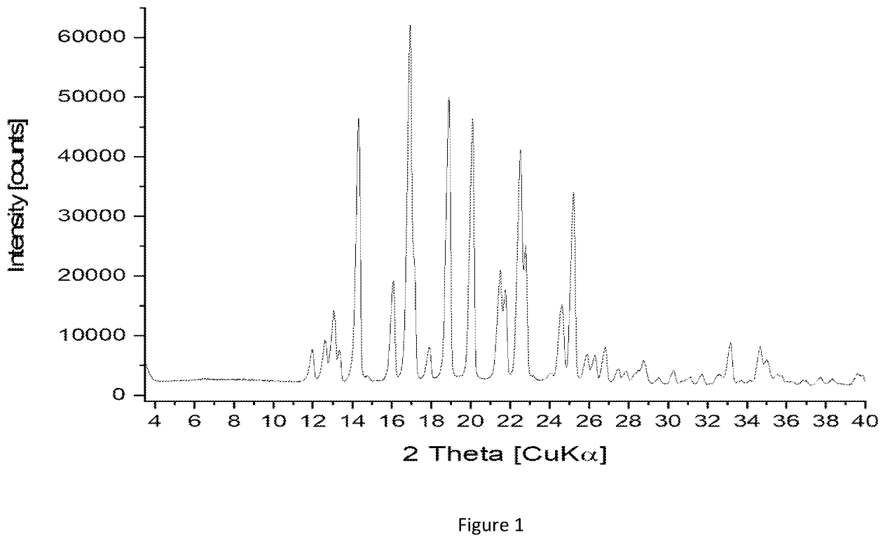

FIG. 2 is an infrared (IR) spectrum of the crystalline form of indaziflam of the present invention from a sample obtained from example 2.

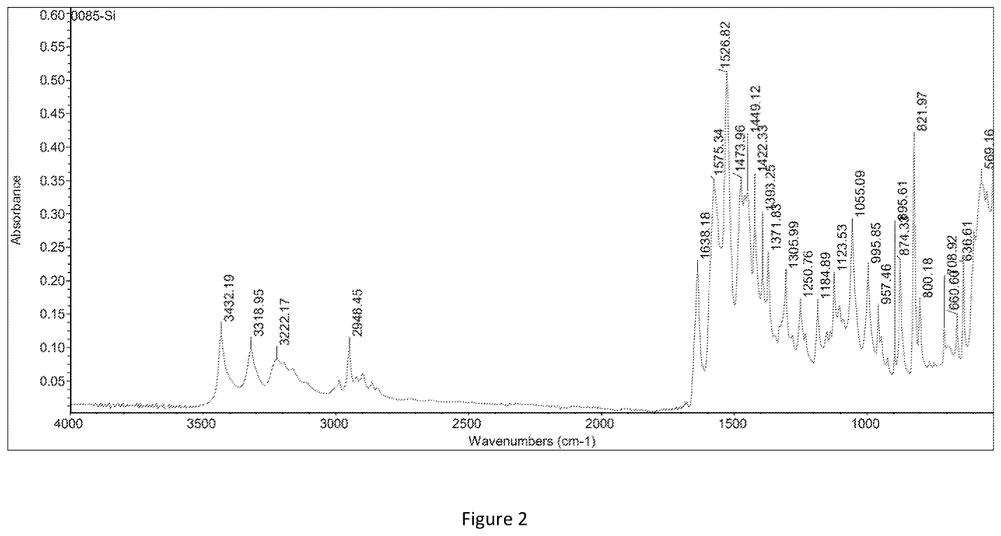

### DETAILED DESCRIPTION OF THE INVENTION

### Definitions

Embodiments of the present invention are discussed in detail below. In describing embodiments, specific terminology is employed for the sake of clarity. However, the invention is not intended to be limited to the specific terminology so selected. While specific exemplary embodiments are discussed, it should be understood that this is done for illustration purposes only. A person skilled in the relevant art will recognize that other components and configurations can be used without parting from the spirit and scope of the invention. While a number of embodiments and features are described herein, it is to be understood that the various features of the invention and aspects of embodiments, even if described separately, may be combined unless mutually exclusive or contrary to the specific description. All references cited herein are incorporated by reference as if each had been individually incorporated.

As used herein, the transitional term “comprising” or “that comprises”, which is synonymous with “including,” or “containing,” is inclusive or open-ended and does not exclude additional, un-recited elements or method steps. However, in each recitation of “comprising” herein, it is intended that the term also encompass, as alternative embodiments, the phrases “consisting essentially of” and “consisting of”, where “consisting of” excludes any element or step not specified and “consisting essentially of” permits the inclusion of additional un-recited elements or steps that do not materially affect the essential or basic and novel characteristics of the composition or method under consideration.

For purposes of sufficiency of disclosure, the “compressibility index” means in the present invention the compressibility index measured according to US Pharmacopoeia method <1174> Powder Flow, revision from 20 Jan. 2023.

Prior to setting forth the present subject matter in detail, it may be helpful to provide definitions of certain terms to be used herein. Unless defined otherwise, all technical and scientific terms used herein have the same meaning as is commonly understood by one of skill in the art to which this subject matter pertains.

As used herein, the term “water immiscible solvent” refers to a solvent that is not water miscible, or hardly miscible, and in particular, the term “water immiscible solvent” is used in this invention to cover any solvent which has a water solubility of less than 1 g/l, measured at 25° C. The term “water miscible solvent” has the opposite meaning to “water immiscible solvent”.

As used herein, the term “aprotic solvent” refers to a solvent which lacks an acidic proton.

As used herein, the term “polar aprotic solvent” refers to a polar solvent which lacks an acidic proton. Polar solvent is a synonym of water miscible solvent. In particular, the term “polar aprotic solvent” is used in this invention to cover any water miscible solvent which lacks an acidic proton and has a dielectric constant of 12 or higher, measured at 25° C.

The term “a” or “an” as used herein includes the singular and the plural, unless specifically stated otherwise. Therefore, the terms “a,” “an” or “at least one” can be used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques. In this regard, used of the term “about” herein specifically includes ±10% from the indicated values in the range. In addition, the endpoints of all ranges directed to the same component or property herein are inclusive of the endpoints, are independently combinable, and include all intermediate points and ranges. Similarly, the ranges and amounts for each element of the technology described herein can be used together with ranges or amounts for any of the other elements.

### Crystalline Form of Indaziflam

The present invention relates to a crystalline form of indaziflam which has a compressibility index lower or equal to 17.

This crystalline form of indaziflam provides a higher flowability than the indaziflam obtained by the methods of the prior art. Flowability is a feature of significant importance to achieve a good manufacturing efficiency and product quality in solid processing industries, to avoid capacity shortfall and production interruption.

The crystalline form of indaziflam may exhibit an X-ray powder diffractogram (XRD) recorded using Cu-Kα radiation at 25° C. with peaks at diffraction angles 2θ±0.2 degree at 14.3, 16.9, 18.9, 20.1, 22.5 and 25.2 degrees.

The crystalline form of indaziflam may exhibit an X-ray powder diffractogram (XRD) recorded using Cu-Kα radiation at 25° C. with peaks at diffraction angles 2θ±0.2 degree at 14.3, 16.9, 18.9, 20.1, 22.5 and 25.2 degrees, and it may have also additional peaks at diffraction angles 2θ±0.2 degree at 13.0, 16.1, 21.5, 24.6 degrees.

The crystalline form of indaziflam may exhibit an infrared (IR) spectrum with characteristic functional peaks at wavenumbers (±2 cm⁻¹) at 569, 822, 1055, 1422, 1449, 1474, 1527, 1575 cm⁻¹.

The crystalline form of indaziflam may exhibit an infrared (IR) spectrum with characteristic functional peaks at wavenumbers (±2 cm⁻¹) at 569, 822, 1055, 1422, 1449, 1474, 1527, 1575 cm, and it may have also additional functional peaks at wavenumbers (±2 cm⁻¹) at 637, 709, 874, 895, 996, 1124, 1306, 1372, 1393, 1638, 2948, 3222, 3319, 3432 cm⁻¹.

The crystalline form of indaziflam may exhibit an X-ray powder diffractogram (XRD) recorded using Cu-Kα radiation at 25° C. with peaks at diffraction angles 2θ±0.2 degree at 14.3, 16.9, 18.9, 20.1, 22.5 and 25.2 degrees, and it may exhibit an infrared (IR) spectrum with characteristic functional peaks at wavenumbers (±2 cm⁻¹) at 569, 822, 1055, 1422, 1449, 1474, 1527, 1575 cm 1. The crystalline form of indaziflam may exhibit an X-ray powder diffractogram (XRD) recorded using Cu-Kα radiation at 25° C. with peaks at diffraction angles 2θ±0.2 degree at 14.3, 16.9, 18.9, 20.1, 22.5 and 25.2 degrees, it may have also additional peaks at diffraction angles 2θ±0.2 degree at 13.0, 16.1, 21.5, 24.6 degrees, it may exhibit an infrared (IR) spectrum with characteristic functional peaks at wavenumbers (±2 cm⁻¹) at 569, 822, 1055, 1422, 1449, 1474, 1527, 1575 cm⁻¹, and it may have also additional functional peaks at wavenumbers (±2 cm⁻¹) at 637, 709, 874, 895, 996, 1124, 1306, 1372, 1393, 1638, 2948, 3222, 3319, 3432 cm⁻¹.

The crystalline form of indaziflam may exhibit a melting point of from 181.4° C. to 183.4° C.

The crystalline form of indaziflam may exhibit a differential scanning calorimetry (DSC) profile having an endothermic melting peak with onset at 182.4° C.±0.2° C. and peak maximum at 182.8° C.±0.2° C.

The crystalline form of indaziflam may exhibit a differential scanning calorimetry (DSC) profile having an endothermic melting peak with onset at 182.4° C.±0.2° C. and peak maximum at 182.8° C.±0.2° C., and it may exhibit an X-ray powder diffractogram (XRD) recorded using Cu-Kα radiation at 25° C. with peaks at diffraction angles 2θ±0.2 degree at 14.3, 16.9, 18.9, 20.1, 22.5 and 25.2 degrees.

The crystalline form of indaziflam may exhibit a differential scanning calorimetry (DSC) profile having an endothermic melting peak with onset at 182.4° C.±0.2° C. and peak maximum at 182.8° C.±0.2° C., it may exhibit an X-ray powder diffractogram (XRD) recorded using Cu-Kα radiation at 25° C. with peaks at diffraction angles 2θ±0.2 degree at 14.3, 16.9, 18.9, 20.1, 22.5 and 25.2 degrees, and the diffractogram may have also additional peaks at diffraction angles 2θ±0.2 degree at 13.0, 16.1, 21.5, 24.6 degrees.

The crystalline form of indaziflam may exhibit a differential scanning calorimetry (DSC) profile having an endothermic melting peak with onset at 182.4° C.±0.2° C. and peak maximum at 182.8° C.±0.2° C., it may exhibit an X-ray powder diffractogram (XRD) recorded using Cu-Kα radiation at 25° C. with peaks at diffraction angles 2θ±0.2 degree at 14.3, 16.9, 18.9, 20.1, 22.5 and 25.2 degrees, and it may exhibit an infrared (IR) spectrum with characteristic functional peaks at wavenumbers (±2 cm⁻¹) at 569, 822, 1055, 1422, 1449, 1474, 1527, 1575 cm⁻¹.

The crystalline form of indaziflam may exhibit a differential scanning calorimetry (DSC) profile having an endothermic melting peak with onset at 182.4° C.±0.2° C. and peak maximum at 182.8° C.±0.2° C., it may exhibit an X-ray powder diffractogram (XRD) recorded using Cu-Kα radiation at 25° C. with peaks at diffraction angles 2θ±0.2 degree at 14.3, 16.9, 18.9, 20.1, 22.5 and 25.2 degrees, the diffractogram may have also additional peaks at diffraction angles 2θ±0.2 degree at 13.0, 16.1, 21.5, 24.6 degrees, it may exhibit an infrared (IR) spectrum with characteristic functional peaks at wavenumbers (±2 cm⁻¹) at 569, 822, 1055, 1422, 1449, 1474, 1527, 1575 cm 1, and it may have also additional functional peaks at wavenumbers (±2 cm⁻¹) at 637, 709, 874, 895, 996, 1124, 1306, 1372, 1393, 1638, 2948, 3222, 3319, 3432 cm⁻¹.

The crystalline form of indaziflam may exhibit a plate morphology.

### Process for the Preparation of a Crystalline Form of Indaziflam

The present invention also relates to a process for the preparation of a crystalline form of indaziflam which has a compressibility index lower or equal to 17, wherein indaziflam is recrystallized in a solvent selected from the group consisting of alcohols, alcohols mixed with water, amides, lactams, esters of carboxylic acids, optionally halogenated aliphatic or aromatic solvents.

As noted above, it has been found that a crystalline form of indaziflam which has a compressibility index lower or equal to 17 exhibits a higher flowability than the indaziflam obtained by the methods of the prior art.

Indaziflam which has a compressibility index lower or equal to 17 may be prepared by any one of the processes for the preparation of indaziflam disclosed in the priority documents with application numbers EP 22182294.3 or EP 22217081.3.

Indaziflam which has a compressibility index lower or equal to 17 may be prepared according to a process which comprises:
a1) a first step which is the preparation of (1R,2S)-1-(bisguanidino)-2,6-dimethylindane or an acid addition salt thereof, by reaction of 1-cyanoguanidine with (1R,2S)-2,6-dimethyl-2,3-dihydro-1H-inden-1-amine, in a mixture of a polar aprotic solvent with a dielectric constant higher than 12.0, when measured at 25° C., and a water immiscible solvent;
or alternatively,
a2) a first step which is the preparation of (1R,2S)-1-(bisguanidino)-2,6-dimethylindane or an acid addition salt thereof, by reaction of 1-cyanoguanidine with (1R,2S)-2,6-dimethyl-2,3-dihydro-1H-inden-1-amine, in a mixture which comprises a trimethylsilyl-containing compound, and an optionally alkyl substituted N-alkyl γ-, δ-, ε-lactam or an optionally alkyl substituted γ-, δ-, ε-lactone;
b) a second step which is the addition of a base and methyl (R)-2-fluoropropanoate to (1R,2S)-1-(bisguanidino)-2,6-dimethylindane or an addition salt thereof, obtained in the first step a1) or a2), and
c) recrystallization of indaziflam obtained in step b) in a solvent selected from the group consisting of alcohols, alcohols mixed with water, amides, lactams, esters of carboxylic acids, optionally halogenated aliphatic or aromatic solvents.

The steps a1), a2) and b) of the process of the invention are carried out under a protective gas atmosphere.

The amount of methyl (R)-2-fluoropropanoate in step b) of the process of the invention may range from 1 to 5 moles, from 1 to 3 moles, from 1 to 2 moles by mole of (1R,2S)-1-(bisguanidino)-2,6-dimethylindane.

The base in step b) of the process of the invention may be selected from the group consisting of an alkali metal, alkaline earth metal or ammonium hydroxide, hydride, alkoxide, carbonate, bicarbonate, phosphate, hydrogen- or dihydrogenphosphate, or a tertiary or aromatic amine or mixtures thereof. The base in step b) of the process of the invention may be selected from the group consisting of an alkali metal or alkaline earth metal hydroxide, alkoxide, carbonate, bicarbonate, or mixtures thereof. The base in step b) of the process of the invention may be selected from the group consisting of an alkali metal hydroxide, methoxide, ethoxide, propoxide, butoxide, carbonate or mixtures thereof.

The amount of the base in step b) of the process of the invention may range from 1 to 5 moles, from 1 to 4 moles, from 1 to 3 moles by mole of (1R,2S)-1-(bisguanidino)-2,6-dimethylindane.

The amount of the base in step b) of the process of the invention may range from 1 to 5 moles by mole of (1R,2S)-1-(bisguanidino)-2,6-dimethylindane and the base may be selected from the group consisting of an alkali metal, alkaline earth metal or ammonium hydroxide, hydride, alkoxide, carbonate, bicarbonate, phosphate, hydrogen- or dihydrogenphosphate, or a tertiary or aromatic amine or mixtures thereof. The amount of the base in step b) of the process of the invention may range from 1 to 4 moles by mole of (1R,2S)-1-(bisguanidino)-2,6-dimethylindane and the base may be selected from the group consisting of an alkali metal or alkaline earth metal hydroxide, alkoxide, carbonate, bicarbonate, or mixtures thereof. The amount of the base in step b) of the process of the invention may range from 1 to 3 moles by mole of (1R,2S)-1-(bisguanidino)-2,6-dimethylindane and the base may be selected from the group consisting of an alkali metal hydroxide, methoxide, ethoxide, propoxide, butoxide, carbonate or mixtures thereof.

The solvent of the reaction in step b) may be any organic solvent. The solvent of the reaction in step b) may be selected from the group consisting of optionally halogen substituted aromatic hydrocarbons, optionally halogen substituted aliphatic hydrocarbons, nitrogen heterocyclic compounds, optionally alkyl substituted cyclic ethers, aliphatic ethers, ethers of aromatic hydrocarbons, nitriles, ketones, esters, amides, alcohols or glycols. The solvent of the reaction in step b) may be selected from the group consisting of toluene, benzene, ethylbenzene, xylenes, cumene, cymenes, mesitylene, biphenyl, chlorobenzene, bromobenzene, o-dichlorobenzene, m-dichlorobenzene, p-dichlorobenzene, chloromethane, dichloromethane, trichloromethane, tetrachloromethane, dichloroethane, trichloroethane, trichloroethylene, tetrachloroethylene, pentane, hexane, heptane, cyclohexane, decalin, quinoline, isoquinoline, quinoxaline, phthalazine, quinazoline, cinnoline, pyridine, pyridazine, pyrimidine, pyrazine, triazines, 1,4-dioxane, tetrahydropyran, methyl tetrahydropyran, tetrahydrofuran, methyl tetrahydrofuran, dibutylether, tert-butyl methyl ether, anisole, acetonitrile, benzonitrile, acetone, butanone, ethyl acetate, n-butyl acetate, hexyl acetate, dimethylformamide or dimethylacetamide, ethanol, methanol, butanol, tert-butanol, propanol, isopropyl alcohol, isoamyl alcohol, phenol, ethylene glycol, propylene glycol, diethylene glycol or dimethoxyethane. The solvent of the reaction in step b) may be selected from the group consisting of toluene, 1,4-dioxane, tetrahydropyran, methyl tetrahydropyran, tetrahydrofuran, methyl tetrahydrofuran, anisole, acetonitrile, dimethylformamide, dimethylacetamide, ethanol, methanol or butanol. The solvent of the reaction in step b) may be ethanol or methanol.

The temperature of step b) of the process of the invention may be from room temperature to the reflux temperature of the solvent in step b).

The acid addition salt of (1R,2S)-1-(bisguanidino)-2,6-dimethylindane in step a1) or a2) may be the salt of an acid such as for example, hydrogen chloride, hydrogen bromide, hydrogen iodide, phosphoric acid, sulfuric acid, nitric acid, carbonic acid, mono- or bifunctional carboxylic acids and hydroxycarboxylic acids, such as acetic acid, oxalic acid, maleic acid, succinic acid, fumaric acid, tartaric acid, citric acid, salicylic acid, sorbic acid or lactic acid, and also sulfonic acids, such as methanesulfonic acid, p-toluenesulfonic acid or 1,5-naphthalenedisulfonic acid.

The process for the preparation of indaziflam may be carried out in one-pot synthesis without isolation of (1R,2S)-1-(bisguanidino)-2,6-dimethylindane or an acid addition salt thereof, or it may be performed in 2 separate steps with isolation of (1R,2S)-1-(bisguanidino)-2,6-dimethylindane or an acid addition salt thereof after step a1) or a2).

In one embodiment, (1R,2S)-1-(bisguanidino)-2,6-dimethylindane or an acid addition salt thereof may be isolated after step a1) or a2) by any conventional method known by the person skilled in the art, including but not limited to filtration, washing the reaction product with a solvent or mixture of solvents in order to dissolve the impurities of (1R,2S)-1-(bisguanidino)-2,6-dimethylindane or an acid addition salt thereof, or by crystallization of (1R,2S)-1-(bisguanidino)-2,6-dimethylindane or an acid addition salt thereof.

In other embodiment, (1R,2S)-1-(bisguanidino)-2,6-dimethylindane or an acid addition salt thereof is not isolated, then, the preparation of indaziflam is carried out in a one-pot synthesis, and the solvent from step a1) or a2) is removed before the reaction of step b) of the process of the invention. The solvent may be removed by any conventional method known by the person skilled in the art, including but not limited to distillation, or distillation under vacuum.

The temperature of the reaction in step a1) of the process of the invention may be in the range from 105° C. to 150° C., from 110° C. to 148° C., from 120° C. to 139° C., from 125° C. to 139° C. The temperature of the reaction in step a1) of the process of the invention may be the temperature of reflux of the mixture of solvents. The mixture of the polar aprotic solvent with a dielectric constant higher than 12.0, when measured at 25° C., and the water immiscible solvent in step a1) may have a boiling point of at least 105° C. The temperature of the reaction in step a1) of the process of the invention may be the temperature of reflux of the mixture of the polar aprotic solvent with a dielectric constant higher than 12.0, when measured at 25° C., and the water immiscible solvent with a boiling point from 105° C. to 150° C., from 110° C. to 148° C., from 120° C. to 139° C., from 125° C. to 139° C. This reaction of step a1) may be carried out at atmospheric pressure or a pressure higher than the atmospheric pressure.

The ratio by weight between the polar aprotic solvent with a dielectric constant higher than 12.0, when measured at 25° C., and the water immiscible solvent may range from 1:1 to 1:5, or from 1:1.5 to 1:3 in the mixture of solvents for the reaction in step a1) of the process of the invention. The ratio by weight between the polar aprotic solvent with a dielectric constant higher than 12.0, when measured at 25° C., and the water immiscible solvent may range from 1:1 to 1:5, or from 1:1.5 to 1:3, and this mixture of solvents in step a1) may have a boiling point of at least 105° C. The ratio by weight between the polar aprotic solvent with a dielectric constant higher than 12.0, when measured at 25° C., and the water immiscible solvent may range from 1:1 to 1:5, or from 1:1.5 to 1:3, and this mixture of solvents in step a1) may have a boiling point from 105° C. to 150° C., from 110° C. to 148° C., from 120° C. to 139° C., from 125° C. to 139° C. The ratio by weight between the polar aprotic solvent with a dielectric constant higher than 12.0, when measured at 25° C., and the water immiscible solvent may range from 1:1 to 1:5, or from 1:1.5 to 1:3, this mixture of solvents in step a1) may have a boiling point from 105° C. to 150° C., from 110° C. to 148° C., from 120° C. to 139° C., from 125° C. to 139° C., and the temperature of the reaction in step a1) of the process of the invention may be the temperature of reflux of the mixture of solvents. This reaction of step a1) may be carried out at atmospheric pressure or a pressure higher than the atmospheric pressure.

The polar aprotic solvent with a dielectric constant higher than 12.0, when measured at 25° C., may be added dropwise or it may be added at once to the mixture of 1-cyanoguanidine with the (1R,2S)-2,6-dimethyl-2,3-dihydro-1H-inden-1-amine and the water immiscible solvent in step a1), or it may be added dropwise together with 1-cyanoguanidine to the mixture of (1R,2S)-2,6-dimethyl-2,3-dihydro-1H-inden-1-amine and the water immiscible solvent in step a1) of the process of the invention.

The polar aprotic solvent with a dielectric constant higher than 12.0, when measured at 25° C., in step a1) may have a dielectric constant higher than 15.0, higher than 20.0, higher than 25.0.

The polar aprotic solvent with a dielectric constant higher than 12.0, when measured at 25° C., may be selected from the group consisting of N,N-dimethylformamide, dimethylacetamide, N-methylpyrrolidone, dimethylsulfoxide, acetonitrile, benzonitrile, nitromethane, nitrobenzene, γ-butyrolactone, sulfolane, nitromethane, tetramethyl urea, propylene carbonate, hexamethylphosphoramide, butanone, methyl isobutyl ketone, acetone, pyridine or N,N-dimethylpropyleneurea in step a1) of the process of the invention. The polar aprotic solvent with a dielectric constant higher than 15.0, when measured at 25° C., in step a1) may be selected from the group consisting of N,N-dimethylformamide, dimethylacetamide, N-methylpyrrolidone, dimethylsulfoxide, acetonitrile, benzonitrile, butanone, methyl isobutyl ketone or acetone. The polar aprotic solvent with a dielectric constant higher than 20.0, when measured at 25° C., in step a1) may be selected from the group consisting of N,N-dimethylformamide, N-methylpyrrolidone or dimethylsulfoxide. The polar aprotic solvent with a dielectric constant higher than 25.0, when measured at 25° C., in step a1) may be N-methylpyrrolidone.

The polar aprotic solvent with a dielectric constant higher than 12.0, when measured at 25° C., may be selected from the group consisting of N,N-dimethylformamide, dimethylacetamide, N-methylpyrrolidone, dimethylsulfoxide, acetonitrile, benzonitrile, nitromethane, nitrobenzene, γ-butyrolactone, sulfolane, nitromethane, tetramethyl t urea, propylene carbonate, hexamethylphosphoramide, butanone, methyl isobutyl ketone, acetone, pyridine or N,N-dimethylpropyleneurea and the ratio by weight between the polar aprotic solvent with a dielectric constant higher than 12.0, when measured at 25° C., and the water immiscible solvent may range from 1:1 to 1:5, or from 1:1.5 to 1:3 in step a1) of the process of the invention. The polar aprotic solvent with a dielectric constant higher than 15.0, when measured at 25° C., may be selected from the group consisting of N,N-dimethylformamide, dimethylacetamide, N-methylpyrrolidone, dimethylsulfoxide, acetonitrile, benzonitrile, butanone, methyl isobutyl ketone or acetone, the ratio by weight between the polar aprotic solvent with a dielectric constant higher than 15.0, when measured at 25° C., and the water immiscible solvent may range from 1:1 to 1:5, or from 1:1.5 to 1:3, and this mixture of solvents may have a boiling point of at least 105° C. in step a1) of the process of the invention. The polar aprotic solvent with a dielectric constant higher than 20.0, when measured at 25° C., may be selected from the group consisting of N,N-dimethylformamide, N-methylpyrrolidone or dimethylsulfoxide, the ratio by weight between the polar aprotic solvent with a dielectric constant higher than 20.0, when measured at 25° C., and the water immiscible solvent may range from 1:1 to 1:5, or from 1:1.5 to 1:3, and this mixture of solvents may have a boiling point from 105° C. to 150° C., from 110° C. to 148° C., from 120° C. to 139° C., from 125° C. to 139° C. in step a1) of the process of the invention. The polar aprotic solvent with a dielectric constant higher than 25.0, when measured at 25° C., may be N-methylpyrrolidone, the ratio by weight between the polar aprotic solvent with a dielectric constant higher than 25.0, when measured at 25° C., and the water immiscible solvent may range from 1:1 to 1:5, or from 1:1.5 to 1:3, this mixture of solvents may have a boiling point from 120° C. to 139° C., from 125° C. to 139° C., and the temperature of the reaction in step a1) of the process of the invention may be the temperature of reflux of the mixture of solvents. This reaction of step a1) may be carried out at atmospheric pressure or a pressure higher than the atmospheric pressure.

The water immiscible solvent may be selected from the group consisting of chlorobenzene, bromobenzene, o-dichlorobenzene, m-dichlorobenzene, p-dichlorobenzene, anisole, toluene, benzene, ethylbenzene, xylenes, cumene, cymenes, mesitylene, biphenyl, decalin, n-butyl acetate, hexyl acetate, white mineral oil, tetrachloroethylene or dibutylether in step a1) of the process of the invention. The water immiscible solvent in step a1) may be selected from the group consisting of chlorobenzene, bromobenzene, o-dichlorobenzene, m-dichlorobenzene, p-dichlorobenzene, anisole, toluene, benzene, ethylbenzene, xylenes, cymenes, n-butyl acetate, hexyl acetate, white mineral oil. The water immiscible solvent in step a1) may be selected from the group consisting of o-chlorobenzene, dichlorobenzene, m-dichlorobenzene, p-dichlorobenzene, anisole, hexyl acetate or white mineral oil. The water immiscible solvent in step a1) may be chlorobenzene.

The water immiscible solvent may be selected from the group consisting of chlorobenzene, bromobenzene, o-dichlorobenzene, m-dichlorobenzene, p-dichlorobenzene, anisole, toluene, benzene, ethylbenzene, xylenes, cumene, cymenes, mesitylene, biphenyl, decalin, n-butyl acetate, hexyl acetate, white mineral oil, tetrachloroethylene or dibutylether, and the polar aprotic solvent with a dielectric constant higher than 12.0, when measured at 25° C., may be selected from the group consisting of N,N-dimethylformamide, dimethylacetamide, N-methylpyrrolidone, dimethylsulfoxide, acetonitrile, benzonitrile, nitromethane, nitrobenzene, γ-butyrolactone, sulfolane, nitromethane, tetramethyl urea, propylene carbonate, hexamethylphosphoramide, butanone, methyl isobutyl ketone, acetone, pyridine or N,N-dimethylpropyleneurea in step a1) of the process of the invention. The water immiscible solvent may be selected from the group consisting of chlorobenzene, bromobenzene, o-dichlorobenzene, m-dichlorobenzene, p-dichlorobenzene, anisole, toluene, benzene, ethylbenzene, xylenes, cymenes, n-butyl acetate, hexyl acetate, white mineral oil, and the polar aprotic solvent with a dielectric constant higher than 15.0, when measured at 25° C., may be selected from the group consisting of N,N-dimethylformamide, dimethylacetamide, N-methylpyrrolidone, dimethylsulfoxide, acetonitrile, benzonitrile, butanone, methyl isobutyl ketone or acetone in step a1) of the process of the invention. The water immiscible solvent may be selected from the group consisting of o-chlorobenzene, dichlorobenzene, m-dichlorobenzene, p-dichlorobenzene, anisole, hexyl acetate or white mineral oil, and the polar aprotic solvent with a dielectric constant higher than 20.0, when measured at 25° C., may be selected from the group consisting of N,N-dimethylformamide, N-methylpyrrolidone or dimethylsulfoxide in step a1) of the process of the invention. The water immiscible solvent may be chlorobenzene, and the polar aprotic solvent with a dielectric constant higher than 25.0, when measured at 25° C., may be N-methylpyrrolidone in step a1) of the process of the invention.

The water immiscible solvent may be selected from the group consisting of chlorobenzene, bromobenzene, o-dichlorobenzene, m-dichlorobenzene, p-dichlorobenzene, anisole, toluene, benzene, ethylbenzene, xylenes, cumene, cymenes, mesitylene, biphenyl, decalin, n-butyl acetate, hexyl acetate, white mineral oil, tetrachloroethylene or dibutylether, and the ratio by weight between the polar aprotic solvent with a dielectric constant higher than 12.0, when measured at 25° C., and the water immiscible solvent may range from 1:1 to 1:5, or from 1:1.5 to 1:3 in step a1) of the process of the invention. The water immiscible solvent may be selected from the group consisting of chlorobenzene, bromobenzene, o-dichlorobenzene, m-dichlorobenzene, p-dichlorobenzene, anisole, toluene, benzene, ethylbenzene, xylenes, cymenes, n-butyl acetate, hexyl acetate, white mineral oil, the ratio by weight between the polar aprotic solvent with a dielectric constant higher than 15.0, when measured at 25° C., and the water immiscible solvent may range from 1:1 to 1:5, or from 1:1.5 to 1:3, and this mixture of solvents may have a boiling point of at least 105° C. in step a1) of the process of the invention. The water immiscible solvent may be selected from the group consisting of o-chlorobenzene, dichlorobenzene, m-dichlorobenzene, p-dichlorobenzene, anisole, hexyl acetate or white mineral oil, the ratio by weight between the polar aprotic solvent with a dielectric constant higher than 20.0, when measured at 25° C., and the water immiscible solvent may range from 1:1 to 1:5, or from 1:1.5 to 1:3, and this mixture of solvents may have a boiling point from 105° C. to 150° C., from 110° C. to 148° C., from 120° C. to 139° C., from 125° C. to 139° C. in step a1) of the process of the invention. The water immiscible solvent may be chlorobenzene, the ratio by weight between the polar aprotic solvent with a dielectric constant higher than 25.0, when measured at 25° C., and the water immiscible solvent may range from 1:1 to 1:5, or from 1:1.5 to 1:3, this mixture of solvents may have a boiling point from 120° C. to 139° C., from 125° C. to 139° C., and the temperature of the reaction in step a1) of the process of the invention may be the temperature of reflux of the mixture of solvents. This reaction of step a1) may be carried out at atmospheric pressure or a pressure higher than the atmospheric pressure.

The water immiscible solvent may be selected from the group consisting of chlorobenzene, bromobenzene, o-dichlorobenzene, m-dichlorobenzene, p-dichlorobenzene, anisole, toluene, benzene, ethylbenzene, xylenes, cumene, cymenes, mesitylene, biphenyl, decalin, n-butyl acetate, hexyl acetate, white mineral oil, tetrachloroethylene or dibutylether, the polar aprotic solvent with a dielectric constant higher than 12.0, when measured at 25° C., may be selected from the group consisting of N,N-dimethylformamide, dimethylacetamide, N-methylpyrrolidone, dimethylsulfoxide, acetonitrile, benzonitrile, nitromethane, nitrobenzene, γ-butyrolactone, sulfolane, nitromethane, tetramethyl urea, propylene carbonate, hexamethylphosphoramide, butanone, methyl isobutyl ketone, acetone, pyridine or N,N-dimethylpropyleneurea, and the ratio by weight between the polar aprotic solvent with a dielectric constant higher than 12.0, when measured at 25° C., and the water immiscible solvent may range from 1:1 to 1:5, or from 1:1.5 to 1:3 in step a1) of the process of the invention. The water immiscible solvent may be selected from the group consisting of chlorobenzene, bromobenzene, o-dichlorobenzene, m-dichlorobenzene, p-dichlorobenzene, anisole, toluene, benzene, ethylbenzene, xylenes, cymenes, n-butyl acetate, hexyl acetate, white mineral oil, the polar aprotic solvent with a dielectric constant higher than 15.0, when measured at 25° C., may be selected from the group consisting of N,N-dimethylformamide, dimethylacetamide, N-methylpyrrolidone, dimethylsulfoxide, acetonitrile, benzonitrile, butanone, methyl isobutyl ketone or acetone, the ratio by weight between the polar aprotic solvent with a dielectric constant higher than 15.0, when measured at 25° C., and the water immiscible solvent may range from 1:1 to 1:5, or from 1:1.5 to 1:3, and this mixture of solvents may have a boiling point of at least 105° C. in step a1) of the process of the invention. The water immiscible solvent may be selected from the group consisting of o-chlorobenzene, dichlorobenzene, m-dichlorobenzene, p-dichlorobenzene, anisole, hexyl acetate or white mineral oil, the polar aprotic solvent with a dielectric constant higher than 20.0, when measured at 25° C., may be selected from the group consisting of N,N-dimethylformamide, N-methylpyrrolidone or dimethylsulfoxide, the ratio by weight between the polar aprotic solvent with a dielectric constant higher than 20.0, when measured at 25° C., and the water immiscible solvent may range from 1:1 to 1:5, or from 1:1.5 to 1:3, and this mixture of solvents may have a boiling point from 105° C. to 150° C., from 110° C. to 148° C., from 120° C. to 139° C., from 125° C. to 139° C. in step a1) of the process of the invention. The water immiscible solvent may be chlorobenzene, the polar aprotic solvent with a dielectric constant higher than 25.0, when measured at 25° C., may be N-methylpyrrolidone, the ratio by weight between the polar aprotic solvent with a dielectric constant higher than 25.0, when measured at 25° C., and the water immiscible solvent may range from 1:1 to 1:5, or from 1:1.5 to 1:3, this mixture of solvents may have a boiling point from 120° C. to 139° C., from 125° C. to 139° C., and the temperature of the reaction in step a1) of the process of the invention may be the temperature of reflux of the mixture of solvents. This reaction in step a1) may be carried out at atmospheric pressure or a pressure higher than the atmospheric pressure.

The temperature of the reaction in step a2) of the process of the invention may range from 60° C. to 100° C., from 65° C. to 100° C., from 70° C. to 100° C. The reaction of step a2) may be carried out at atmospheric pressure or a pressure higher than the atmospheric pressure.

The trimethylsilyl-containing compound, and the optionally alkyl substituted N-alkyl γ-, δ-, ε-lactam or the optionally alkyl substituted γ-, δ-, ε-lactone may be added dropwise or they may be added at once to the mixture of 1-cyanoguanidine with (1R,2S)-2,6-dimethyl-2,3-dihydro-1H-inden-1-amine in step a2) of the process of the invention. The trimethylsilyl-containing compound, and the optionally alkyl substituted N-alkyl γ-, δ-, ε-lactam or the optionally alkyl substituted γ-, δ-, ε-lactone may be added together or separately in step a2) of the process of the invention.

The mole ratio between the trimethylsilyl-containing compound, and the optionally alkyl substituted N-alkyl γ-, δ-, ε-lactam or the optionally alkyl substituted γ-, δ-, ε-lactone may range from 1:1 to 1:20, or from 1:1.5 to 1:15 in step a2) of the process of the invention. The mole ratio between the trimethylsilyl-containing compound, and the optionally alkyl substituted N-alkyl γ-, δ-, ε-lactam or the optionally alkyl substituted γ-, δ-, ε-lactone may range from 1:1 to 1:20, or from 1:1.5 to 1:15 and the temperature of the reaction may range from 60° C. to 100° C. in step a2) of the process of the invention. The reaction of step a2) may be carried out at atmospheric pressure or a pressure higher than the atmospheric pressure.

The amount of the trimethylsilyl-containing compound may be at least 1 mole, at least 1.02 moles, at least 1.05 moles by mole of (1R,2S)-2,6-dimethyl-2,3-dihydro-1H-inden-1-amine in step a2) of the process of the invention. The amount of the trimethylsilyl-containing compound may be at least 1 mole, at least 1.02 moles, at least 1.05 moles by mole of (1R,2S)-2,6-dimethyl-2,3-dihydro-1H-inden-1-amine; and the mole ratio between the trimethylsilyl-containing compound, and the optionally alkyl substituted N-alkyl γ-, δ-, ε-lactam or the optionally alkyl substituted γ-, δ-, ε-lactone may range from 1:1 to 1:20, or from 1:1.5 to 1:15 in step a2) of the process of the invention. The amount of the trimethylsilyl-containing compound may be at least 1 mole, at least 1.02 moles, at least 1.05 moles by mole of (1R,2S)-2,6-dimethyl-2,3-dihydro-1H-inden-1-amine; the mole ratio between the trimethylsilyl-containing compound, and the optionally alkyl substituted N-alkyl γ-, δ-, ε-lactam or the optionally alkyl substituted γ-, δ-, ε-lactone may range from 1:1 to 1:20, or from 1:1.5 to 1:15; and the temperature of the reaction may range from 60° C. to 100° C., from 65° C. to 100° C., from 70° C. to 100° C. in step a2) of the process of the invention. The amount of the trimethylsilyl-containing compound may be at least 1.05 moles by mole of (1R,2S)-2,6-dimethyl-2,3-dihydro-1H-inden-1-amine; the mole ratio between the trimethylsilyl-containing compound, and the optionally alkyl substituted N-alkyl γ-, δ-, ε-lactam or the optionally alkyl substituted γ-, δ-, ε-lactone may range from 1:1 to 1:20, or from 1:1.5 to 1:15; the mole ratio between the trimethylsilyl-containing compound, and the 1-cyanoguanidine may range from 0.6:1 to 1:0.6; and the temperature of the reaction may range from 60° C. to 100° C. in step a2) of the process of the invention. The reaction of step a2) may be carried out at atmospheric pressure or a pressure higher than the atmospheric pressure.

The amount of the optionally alkyl substituted N-alkyl γ-, δ-, ε-lactam or the optionally alkyl substituted γ-, δ-, ε-lactone may be at least 1 mole, at least 1.5 moles, at least 2 moles by mole of (1R,2S)-2,6-dimethyl-2,3-dihydro-1H-inden-1-amine in step a2) of the process of the invention. The amount of the optionally alkyl substituted N-alkyl γ-, δ-, ε-lactam or the optionally alkyl substituted γ-, δ-, ε-lactone may be at least 1 mole, at least 1.5 moles, at least 2 moles by mole of (1R,2S)-2,6-dimethyl-2,3-dihydro-1H-inden-1-amine; and the mole ratio between the trimethylsilyl-containing compound, and the optionally alkyl substituted N-alkyl γ-, δ-, ε-lactam or the optionally alkyl substituted γ-, δ-, ε-lactone may range from 1:1 to 1:20, or from 1:1.5 to 1:15 in step a2) of the process of the invention. The amount of the optionally alkyl substituted N-alkyl γ-, δ-, ε-lactam or the optionally alkyl substituted γ-, δ-, ε-lactone may be at least 1 mole, at least 1.5 moles, at least 2 moles by mole of (1R,2S)-2,6-dimethyl-2,3-dihydro-1H-inden-1-amine; the mole ratio between the trimethylsilyl-containing compound, and the optionally alkyl substituted N-alkyl γ-, δ-, ε-lactam or the optionally alkyl substituted γ-, δ-, ε-lactone may range from 1:1 to 1:20, or from 1:1.5 to 1:15; and the temperature of the reaction may range from 60° C. to 100° C., from 65° C. to 100° C., from 70° C. to 100° C. in step a2) of the process of the invention. The amount of the optionally alkyl substituted N-alkyl γ-, δ-, ε-lactam or the optionally alkyl substituted γ-, δ-, ε-lactone may be at least 1 mole, at least 1.5 moles, at least 2 moles by mole of (1R,2S)-2,6-dimethyl-2,3-dihydro-1H-inden-1-amine; the mole ratio between the trimethylsilyl-containing compound, and the optionally alkyl substituted N-alkyl γ-, δ-, ε-lactam or the optionally alkyl substituted γ-, δ-, ε-lactone may range from 1:1 to 1:20, or from 1:1.5 to 1:15; the mole ratio between the optionally alkyl substituted N-alkyl γ-, δ-, ε-lactam or the optionally alkyl substituted γ-, δ-, ε-lactone, and the 1-cyanoguanidine may range from 1:1 to 5:1, from 1.5:1 to 3:1; and the temperature of the reaction may range from 60° C. to 100° C., from 65° C. to 100° C., from 70° C. to 100° C. in step a2) of the process of the invention. The amount of the optionally alkyl substituted N-alkyl γ-, δ-, ε-lactam or the optionally alkyl substituted γ-, δ-, ε-lactone may be at least 1 mole, at least 1.5 moles, at least 2 moles by mole of (1R,2S)-2,6-dimethyl-2,3-dihydro-1H-inden-1-amine; the mole ratio between the trimethylsilyl-containing compound, and the optionally alkyl substituted N-alkyl γ-, δ-, ε-lactam or the optionally alkyl substituted γ-, δ-, ε-lactone may range from 1:1 to 1:20, or from 1:1.5 to 1:15; the mole ratio between the optionally alkyl substituted N-alkyl γ-, δ-, ε-lactam or the optionally alkyl substituted γ-, δ-, ε-lactone, and the 1-cyanoguanidine may range from 1:1 to 5:1, from 1.5:1 to 3:1; the mole ratio between the trimethylsilyl-containing compound, and the 1-cyanoguanidine may range from 0.6:1 to 1:0.6, from 0.8:1 to 1:0.8; and the temperature of the reaction may range from 60° C. to 100° C., from 65° C. to 100° C., from 70° C. to 100° C. in step a2) of the process of the invention. The amount of the optionally alkyl substituted N-alkyl γ-, δ-, ε-lactam or the optionally alkyl substituted γ-, δ-, ε-lactone may be at least 1 mole by mole of (1R,2S)-2,6-dimethyl-2,3-dihydro-1H-inden-1-amine; the mole ratio between the trimethylsilyl-containing compound, and the optionally alkyl substituted N-alkyl γ-, δ-, ε-lactam or the optionally alkyl substituted γ-, δ-, ε-lactone may range from 1:1 to 1:20, or from 1:1.5 to 1:15; the mole ratio between the optionally alkyl substituted N-alkyl γ-, δ-, ε-lactam or the optionally alkyl substituted γ-, δ-, ε-lactone, and the 1-cyanoguanidine may range from 1:1 to 5:1; the mole ratio between the trimethylsilyl-containing compound, and the 1-cyanoguanidine may range from 0.6:1 to 1:0.6; the amount of the trimethylsilyl-containing compound may be at least 1.05 moles by mole of (1R,2S)-2,6-dimethyl-2,3-dihydro-1H-inden-1-amine; and the temperature of the reaction may range from 60° C. to 100° C. in step a2) of the process of the invention. The reaction of step a2) may be carried out at atmospheric pressure or a pressure higher than the atmospheric pressure.

The trimethylsilyl-containing compound may be selected from the group consisting of trimethylsilyl halide, trimethylsilyl haloalkylsulfonate, trimetylsilyl halosulfonate, trimethylsilyl azide, trimethylsilyl cyanide, hexamethyldisilane, optionally halogen substituted alkyl trimethylsilane, trimethylsilylalkyne, tris(trimethylsilyl)silane, tris(trimethylsilyl)methane, tetrakis(trimethylsilyl)silane, optionally halogen substituted (trimethylsilyl)alkylamide, or secondary or tertiary (trimethylsilyl)amines in step a2) of the process of the invention. The trimethylsilyl-containing compound may be selected from the group consisting of trimethylsilyl chloride, trimethylsilyl iodide, trimethylsilyl bromide, trimetylsilyl trifluoromethanesulfonate, trimethylsilyl chlorosulfonate, trimethylsilyl azide, trimethylsilyl cyanide, hexamethyldisilane, tetrametylsilane, (trimethylsilyl)methyl chloride, 1-(trimethylsilyl)propyne, trimethylsilylacetylene, tris(trimethylsilyl)silane, tris(trimethylsilyl)methane, tetrakis(trimethylsilyl)silane, N,O-bis(trimethylsilyl)acetamide, N,O-bis(trimethylsilyl)trifluoroacetamide, N-methyl-N-(trimethylsilyl)trifluoroacetamide, N-(trimethylsilyl)diethylamine, bis(trimethylsilyl)amine or tris(trimethylsilyl)amine in step a2) of the process of the invention. The trimethylsilyl-containing compound may be selected from the group consisting of trimethylsilyl chloride, trimethylsilyl iodide, trimethylsilyl bromide, trimetylsilyl trifluoromethanesulfonate, N,O-bis(trimethylsilyl)acetamide, N,O-bis(trimethylsilyl)trifluoroacetamide, N-methyl-N-(trimethylsilyl)trifluoroacetamide, N-(trimethylsilyl)diethylamine, bis(trimethylsilyl)amine or tris(trimethylsilyl)amine in step a2) of the process of the invention. The trimethylsilyl-containing compound may be trimethylsilyl chloride or trimetylsilyl trifluoromethanesulfonate in step a2) of the process of the invention.

The trimethylsilyl-containing compound may be selected from the group consisting of trimethylsilyl halide, trimethylsilyl haloalkylsulfonate, trimetylsilyl halosulfonate, trimethylsilyl azide, trimethylsilyl cyanide, hexamethyldisilane, optionally halogen substituted alkyl trimethylsilane, trimethylsilylalkyne, tris(trimethylsilyl)silane, tris(trimethylsilyl)methane, tetrakis(trimethylsilyl)silane, optionally halogen substituted (trimethylsilyl)alkylamide, or secondary or tertiary (trimethylsilyl)amines; and the temperature of the reaction may range from 60° C. to 100° C., from 65° C. to 100° C., from 70° C. to 100° C. in step a2) of the process of the invention. The trimethylsilyl-containing compound may be selected from the group consisting of trimethylsilyl chloride, trimethylsilyl iodide, trimethylsilyl bromide, trimetylsilyl trifluoromethanesulfonate, trimethylsilyl chlorosulfonate, trimethylsilyl azide, trimethylsilyl cyanide, hexamethyldisilane, tetrametylsilane, (trimethylsilyl)methyl chloride, 1-(trimethylsilyl)propyne, trimethylsilylacetylene, tris(trimethylsilyl)silane, tris(trimethylsilyl)methane, tetrakis(trimethylsilyl)silane, N,O-bis(trimethylsilyl)acetamide, N,O-bis(trimethylsilyl)trifluoroacetamide, N-methyl-N-(trimethylsilyl)trifluoroacetamide, N-(trimethylsilyl)diethylamine, bis(trimethylsilyl)amine or tris(trimethylsilyl)amine; the mole ratio between the trimethylsilyl-containing compound, and the optionally alkyl substituted N-alkyl γ-, δ-, ε-lactam or the optionally alkyl substituted γ-, δ-, ε-lactone may range from 1:1 to 1:20, or from 1:1.5 to 1:15; and the temperature of the reaction may range from 60° C. to 100° C., from 65° C. to 100° C., from 70° C. to 100° C. in step a2) of the process of the invention. The trimethylsilyl-containing compound may be selected from the group consisting of trimethylsilyl chloride, trimethylsilyl iodide, trimethylsilyl bromide, trimetylsilyl trifluoromethanesulfonate, N,O-bis(trimethylsilyl)acetamide, N,O-bis(trimethylsilyl)trifluoroacetamide, N-methyl-N-(trimethylsilyl)trifluoroacetamide, N-(trimethylsilyl)diethylamine, bis(trimethylsilyl)amine or tris(trimethylsilyl)amine; the mole ratio between the trimethylsilyl-containing compound, and the optionally alkyl substituted N-alkyl γ-, δ-, ε-lactam or the optionally alkyl substituted γ-, δ-, ε-lactone may range from 1:1 to 1:20, or from 1:1.5 to 1:15; the amount of the trimethylsilyl-containing compound may be at least 1 mole, at least 1.02 moles, at least 1.05 moles by mole of (1R,2S)-2,6-dimethyl-2,3-dihydro-1H-inden-1-amine; and the temperature of the reaction may range from 60° C. to 100° C., from 65° C. to 100° C. in step a2) of the process of the invention. The trimethylsilyl-containing compound may be trimethylsilyl chloride or trimetylsilyl trifluoromethanesulfonate; the mole ratio between the trimethylsilyl-containing compound, and the optionally alkyl substituted N-alkyl γ-, δ-, ε-lactam or the optionally alkyl substituted γ-, δ-, ε-lactone may range from 1:1.5 to 1:15; the amount of the trimethylsilyl-containing compound may be at least 1.05 moles by mole of (1R,2S)-2,6-dimethyl-2,3-dihydro-1H-inden-1-amine; the mole ratio between the trimethylsilyl-containing compound, and the 1-cyanoguanidine may range from 0.6:1 to 1:0.6; and the temperature of the reaction may range from 60° C. to 100° C. in step a2) of the process of the invention. The reaction of step a2) may be carried out at atmospheric pressure or a pressure higher than the atmospheric pressure.

The optionally alkyl substituted N-alkyl γ-, δ-, ε-lactam or the optionally alkyl substituted γ-, δ-, ε-lactone may be selected from the group consisting of optionally alkyl substituted N—(C₁-C₁₈) alkyl pyrrolidones, optionally alkyl substituted N—(C₁-C₄) alkyl 2-piperidones, optionally alkyl substituted N—(C₁-C₄) alkyl caprolactams, optionally alkyl substituted γ-butyrolactones, optionally alkyl substituted δ-valerolactones, optionally alkyl substituted ε-caprolactones, in step a2) of the process of the invention. The optionally alkyl substituted N-alkyl γ-, δ-, ε-lactam or the optionally alkyl substituted γ-, δ-, ε-lactone may be selected from the group consisting of N-methylpyrrolidone, N-ethyl-2-pyrrolidone, N-propyl-2-pyrrolidone, N-butyl-2-pyrrolidone, 1-tert-butylpyrrolidin-2-one, 1-pentylpyrrolidin-2-one, N-hexyl-2-pyrrolidinone, 1-octyl-2-pyrrolidone, N-decyl-2-pyrrolidone, 1-dodecyl-2-pyrrolidinone, 1-tetradecyl-2-pyrrolidinone, 5-methyl-1-tetradecyl-2-pyrrolidinone, 1-octadecyl-2-pyrrolidinone, 3-methyl-1-octadecyl-2-pyrrolidinone, N-methyl-2-piperidone, 1-ethyl-2-piperidinone, 1-propyl-2-piperidinone, 1-butyl-2-piperidinone, N-methyl caprolactam, N-ethyl caprolactam, γ-butyrolactone, γ-valerolactone, γ-caprolactone, γ-octalactone, γ-nonalactone, γ-decalactone, γ-undecalactone, δ-valerolactone, δ-octalactone, δ-decalactone, δ-undecalactone, ε-caprolactone, ε-decalactone or ε-dodecalactone in step a2) of the process of the invention. The optionally alkyl substituted N-alkyl γ-, δ-, ε-lactam or the optionally alkyl substituted γ-, δ-, ε-lactone may be selected from the group consisting of N-methylpyrrolidone, N-ethyl-2-pyrrolidone, N-propyl-2-pyrrolidone, N-butyl-2-pyrrolidone, N-methyl-2-piperidone, N-methyl caprolactam, N-ethyl caprolactam, γ-butyrolactone, δ-valerolactone or ε-caprolactone in step a2) of the process of the invention. The optionally alkyl substituted N-alkyl γ-, δ-, ε-lactam or the optionally alkyl substituted γ-, δ-, ε-lactone may be N-methylpyrrolidone in step a2) of the process of the invention.

The optionally alkyl substituted N-alkyl γ-, δ-, ε-lactam or the optionally alkyl substituted γ-, δ-, ε-lactone may be selected from the group consisting of optionally alkyl substituted N—(C₁-C₁₈) alkyl pyrrolidones, optionally alkyl substituted N—(C₁-C₄) alkyl 2-piperidones, optionally alkyl substituted N—(C₁-C₄) alkyl caprolactams, optionally alkyl substituted γ-butyrolactones, optionally alkyl substituted δ-valerolactones, optionally alkyl substituted ε-caprolactones; and the trimethylsilyl-containing compound may be selected from the group consisting of trimethylsilyl halide, trimethylsilyl haloalkylsulfonate, trimetylsilyl halosulfonate, trimethylsilyl azide, trimethylsilyl cyanide, hexamethyldisilane, optionally halogen substituted alkyl trimethylsilane, trimethylsilylalkyne, tris(trimethylsilyl)silane, tris(trimethylsilyl)methane, tetrakis(trimethylsilyl)silane, optionally halogen substituted (trimethylsilyl)alkylamide, or secondary or tertiary (trimethylsilyl)amines, in step a2) of the process of the invention. The optionally alkyl substituted N-alkyl γ-, δ-, ε-lactam or the optionally alkyl substituted γ-, δ-, ε-lactone may be selected from the group consisting of N-methylpyrrolidone, N-ethyl-2-pyrrolidone, N-propyl-2-pyrrolidone, N-butyl-2-pyrrolidone, 1-tert-butylpyrrolidin-2-one, 1-pentylpyrrolidin-2-one, N-hexyl-2-pyrrolidinone, 1-octyl-2-pyrrolidone, N-decyl-2-pyrrolidone, 1-dodecyl-2-pyrrolidinone, 1-tetradecyl-2-pyrrolidinone, 5-methyl-1-tetradecyl-2-pyrrolidinone, 1-octadecyl-2-pyrrolidinone, 3-methyl-1-octadecyl-2-pyrrolidinone, N-methyl-2-piperidone, 1-ethyl-2-piperidinone, 1-propyl-2-piperidinone, 1-butyl-2-piperidinone, N-methyl caprolactam, N-ethyl caprolactam, γ-butyrolactone, γ-valerolactone, γ-caprolactone, γ-octalactone, γ-nonalactone, γ-decalactone, γ-undecalactone, δ-valerolactone, δ-octalactone, δ-decalactone, δ-undecalactone, ε-caprolactone, ε-decalactone or ε-dodecalactone; the trimethylsilyl-containing compound may be selected from the group consisting of trimethylsilyl chloride, trimethylsilyl iodide, trimethylsilyl bromide, trimetylsilyl trifluoromethanesulfonate, trimethylsilyl chlorosulfonate, trimethylsilyl azide, trimethylsilyl cyanide, hexamethyldisilane, tetrametylsilane, (trimethylsilyl)methyl chloride, 1-(trimethylsilyl)propyne, trimethylsilylacetylene, tris(trimethylsilyl)silane, tris(trimethylsilyl)methane, tetrakis(trimethylsilyl)silane, N,O-bis(trimethylsilyl)acetamide, N,O-bis(trimethylsilyl)trifluoroacetamide, N-methyl-N-(trimethylsilyl)trifluoroacetamide, N-(trimethylsilyl)diethylamine, bis(trimethylsilyl)amine or tris(trimethylsilyl)amine; and the temperature of the reaction may range from 60° C. to 100° C., from 65° C. to 100° C., from 70° C. to 100° C. in step a2) of the process of the invention. The optionally alkyl substituted N-alkyl γ-, δ-, ε-lactam or the optionally alkyl substituted γ-, δ-, ε-lactone may be selected from the group consisting of N-methylpyrrolidone, N-ethyl-2-pyrrolidone, N-propyl-2-pyrrolidone, N-butyl-2-pyrrolidone, N-methyl-2-piperidone, N-methyl caprolactam, N-ethyl caprolactam, γ-butyrolactone, δ-valerolactone or ε-caprolactone; the trimethylsilyl-containing compound may be selected from the group consisting of trimethylsilyl chloride, trimethylsilyl iodide, trimethylsilyl bromide, trimetylsilyl trifluoromethanesulfonate, N,O-bis(trimethylsilyl)acetamide, N,O-bis(trimethylsilyl)trifluoroacetamide, N-methyl-N-(trimethylsilyl)trifluoroacetamide, N-(trimethylsilyl)diethylamine, bis(trimethylsilyl)amine or tris(trimethylsilyl)amine; the mole ratio between the trimethylsilyl-containing compound, and the optionally alkyl substituted N-alkyl γ-, δ-, ε-lactam or the optionally alkyl substituted γ-, δ-, ε-lactone may range from 1:1 to 1:20, or from 1:1.5 to 1:15; and the temperature of the reaction may range from 60° C. to 100° C., from 65° C. to 100° C. in step a2) of the process of the invention. The optionally alkyl substituted N-alkyl γ-, δ-, ε-lactam or the optionally alkyl substituted γ-, δ-, ε-lactone may be N-methylpyrrolidone; the trimethylsilyl-containing compound may be trimethylsilyl chloride or trimethylsilyl trifluoromethanesulfonate; the mole ratio between the trimethylsilyl-containing compound, and the optionally alkyl substituted N-alkyl γ-, δ-, ε-lactam or the optionally alkyl substituted γ-, δ-, ε-lactone may range from 1:1 to 1:20, or from 1:1.5 to 1:15; the mole ratio between the optionally alkyl substituted N-alkyl γ-, δ-, ε-lactam or the optionally alkyl substituted γ-, δ-, ε-lactone, and the 1-cyanoguanidine may range from 1:1 to 5:1; and the temperature of the reaction may range from 60° C. to 100° C. in step a2) of the process of the invention. The reaction of step a2) may be carried out at atmospheric pressure or a pressure higher than the atmospheric pressure.

In one embodiment, the step a2) for the preparation of (1R,2S)-1-(bisguanidino)-2,6-dimethylindane or an acid addition salt thereof may optionally contain an aprotic solvent. The aprotic solvent may be selected from the group consisting of optionally halogen substituted aromatic hydrocarbons, optionally halogen substituted aliphatic hydrocarbons, nitrogen heterocyclic compounds, optionally alkyl substituted cyclic ethers, aliphatic ethers, ethers of aromatic hydrocarbons, nitriles, ketones, esters or tertiary amides, in step a2) of the process of the invention. The aprotic solvent may be selected from the group consisting of toluene, benzene, ethylbenzene, xylenes, cumene, cymenes, mesitylene, biphenyl, chlorobenzene, bromobenzene, o-dichlorobenzene, m-dichlorobenzene, p-dichlorobenzene, chloromethane, dichloromethane, trichloromethane, tetrachloromethane, dichloroethane, trichloroethane, trichloroethylene, tetrachloroethylene, pentane, hexane, heptane, cyclohexane, decalin, quinoline, isoquinoline, quinoxaline, phthalazine, quinazoline, cinnoline, pyridine, pyridazine, pyrimidine, pyrazine, triazines, 1,4-dioxane, tetrahydropyran, methyl tetrahydropyran, tetrahydrofuran, methyl tetrahydrofuran, dibutylether, tert-butyl methyl ether, anisole, acetonitrile, benzonitrile, acetone, butanone, ethyl acetate, n-butyl acetate, hexyl acetate, dimethylformamide or dimethylacetamide in step a2) of the process of the invention. The aprotic solvent may be selected from the group consisting of toluene, benzene, ethylbenzene, xylenes, cumene, cymenes, chlorobenzene, bromobenzene, o-dichlorobenzene, m-dichlorobenzene, p-dichlorobenzene, trichloromethane, tetrachloromethane, dichloroethane, trichloroethane, trichloroethylene, tetrachloroethylene, hexane, cyclohexane, quinoline, pyridine, 1,4-dioxane, tetrahydropyran, methyl tetrahydropyran, tetrahydrofuran, methyl tetrahydrofuran, anisole, acetonitrile, benzonitrile, acetone, ethyl acetate, dimethylformamide or dimethylacetamide in step a2) of the process of the invention. The aprotic solvent may be selected from the group consisting of toluene, chlorobenzene, 1,4-dioxane, tetrahydropyran, methyl tetrahydropyran, tetrahydrofuran, methyl tetrahydrofuran, anisole or acetonitrile in step a2) of the process of the invention.

The aprotic solvent may be selected from the group consisting of optionally halogen substituted aromatic hydrocarbons, optionally halogen substituted aliphatic hydrocarbons, nitrogen heterocyclic compounds, optionally alkyl substituted cyclic ethers, aliphatic ethers, ethers of aromatic hydrocarbons, nitriles, ketones, esters or tertiary amides; and the optionally alkyl substituted N-alkyl γ-, δ-, ε-lactam or the optionally alkyl substituted γ-, δ-, ε-lactone may be selected from the group consisting of optionally alkyl substituted N—(C₁-C₁₈) alkyl pyrrolidones, optionally alkyl substituted N—(C₁-C₄) alkyl 2-piperidones, optionally alkyl substituted N—(C₁-C₄) alkyl caprolactams, optionally alkyl substituted γ-butyrolactones, optionally alkyl substituted δ-valerolactones, optionally alkyl substituted ε-caprolactones, in step a2) of the process of the invention. The aprotic solvent may be selected from the group consisting of toluene, benzene, ethylbenzene, xylenes, cumene, cymenes, mesitylene, biphenyl, chlorobenzene, bromobenzene, o-dichlorobenzene, m-dichlorobenzene, p-dichlorobenzene, chloromethane, dichloromethane, trichloromethane, tetrachloromethane, dichloroethane, trichloroethane, trichloroethylene, tetrachloroethylene, pentane, hexane, heptane, cyclohexane, decalin, quinoline, isoquinoline, quinoxaline, phthalazine, quinazoline, cinnoline, pyridine, pyridazine, pyrimidine, pyrazine, triazines, 1,4-dioxane, tetrahydropyran, methyl tetrahydropyran, tetrahydrofuran, methyl tetrahydrofuran, dibutylether, tert-butyl methyl ether, anisole, acetonitrile, benzonitrile, acetone, butanone, ethyl acetate, n-butyl acetate, hexyl acetate, dimethylformamide or dimethylacetamide; the optionally alkyl substituted N-alkyl γ-, δ-, ε-lactam or the optionally alkyl substituted γ-, δ-, ε-lactone may be selected from the group consisting of N-methylpyrrolidone, N-ethyl-2-pyrrolidone, N-propyl-2-pyrrolidone, N-butyl-2-pyrrolidone, 1-tert-butylpyrrolidin-2-one, 1-pentylpyrrolidin-2-one, N-hexyl-2-pyrrolidinone, 1-octyl-2-pyrrolidone, N-decyl-2-pyrrolidone, 1-dodecyl-2-pyrrolidinone, 1-tetradecyl-2-pyrrolidinone, 5-methyl-1-tetradecyl-2-pyrrolidinone, 1-octadecyl-2-pyrrolidinone, 3-methyl-1-octadecyl-2-pyrrolidinone, N-methyl-2-piperidone, 1-ethyl-2-piperidinone, 1-propyl-2-piperidinone, 1-butyl-2-piperidinone, N-methyl caprolactam, N-ethyl caprolactam, γ-butyrolactone, γ-valerolactone, γ-caprolactone, γ-octalactone, γ-nonalactone, γ-decalactone, γ-undecalactone, δ-valerolactone, δ-octalactone, δ-decalactone, δ-undecalactone, ε-caprolactone, ε-decalactone or ε-dodecalactone; and the trimethylsilyl-containing compound may be selected from the group consisting of trimethylsilyl chloride, trimethylsilyl iodide, trimethylsilyl bromide, trimetylsilyl trifluoromethanesulfonate, trimethylsilyl chlorosulfonate, trimethylsilyl azide, trimethylsilyl cyanide, hexamethyldisilane, tetrametylsilane, (trimethylsilyl)methyl chloride, 1-(trimethylsilyl)propyne, trimethylsilylacetylene, tris(trimethylsilyl)silane, tris(trimethylsilyl)methane, tetrakis(trimethylsilyl)silane, N,O-bis(trimethylsilyl)acetamide, N,O-bis(trimethylsilyl)trifluoroacetamide, N-methyl-N-(trimethylsilyl)trifluoroacetamide, N-(trimethylsilyl)diethylamine, bis(trimethylsilyl)amine or tris(trimethylsilyl)amine in step a2) of the process of the invention. The aprotic solvent may be selected from the group consisting of toluene, benzene, ethylbenzene, xylenes, cumene, cymenes, chlorobenzene, bromobenzene, o-dichlorobenzene, m-dichlorobenzene, p-dichlorobenzene, trichloromethane, tetrachloromethane, dichloroethane, trichloroethane, trichloroethylene, tetrachloroethylene, hexane, cyclohexane, quinoline, pyridine, 1,4-dioxane, tetrahydropyran, methyl tetrahydropyran, tetrahydrofuran, methyl tetrahydrofuran, anisole, acetonitrile, benzonitrile, acetone, ethyl acetate, dimethylformamide or dimethylacetamide; the optionally alkyl substituted N-alkyl γ-, δ-, ε-lactam or the optionally alkyl substituted γ-, δ-, ε-lactone may be selected from the group consisting of N-methylpyrrolidone, N-ethyl-2-pyrrolidone, N-propyl-2-pyrrolidone, N-butyl-2-pyrrolidone, N-methyl-2-piperidone, N-methyl caprolactam, N-ethyl caprolactam, γ-butyrolactone, δ-valerolactone or ε-caprolactone; the trimethylsilyl-containing compound may be selected from the group consisting of trimethylsilyl chloride, trimethylsilyl iodide, trimethylsilyl bromide, trimetylsilyl trifluoromethanesulfonate, N,O-bis(trimethylsilyl)acetamide, N,O-bis(trimethylsilyl)trifluoroacetamide, N-methyl-N-(trimethylsilyl)trifluoroacetamide, N-(trimethylsilyl)diethylamine, bis(trimethylsilyl)amine or tris(trimethylsilyl)amine; and the temperature of the reaction may range from 60° C. to 100° C., from 65° C. to 100° C. in step a2) of the process of the invention. The aprotic solvent may be selected from the group consisting of toluene, chlorobenzene, 1,4-dioxane, tetrahydropyran, methyl tetrahydropyran, tetrahydrofuran, methyl tetrahydrofuran, anisole or acetonitrile; the optionally alkyl substituted N-alkyl γ-, δ-, ε-lactam or the optionally alkyl substituted γ-, δ-, ε-lactone may be N-methylpyrrolidone; the trimethylsilyl-containing compound may be trimethylsilyl chloride or trimethyl trifluoromethanesulfonate; the mole ratio between the trimethylsilyl-containing compound, and the optionally alkyl substituted N-alkyl γ-, δ-, ε-lactam or the optionally alkyl substituted γ-, δ-, ε-lactone may range from 1:1 to 1:20; and the temperature of the reaction may range from 60° C. to 100° C. in step a2) of the process of the invention. The reaction of step a2) may be carried out at atmospheric pressure or a pressure higher than the atmospheric pressure.

Indaziflam obtained according to step b) of the preparation process is recrystallized in step c) in a solvent that may be selected from the group consisting of alcohols, alcohols mixed with water, amides, lactams, esters of carboxylic acids, optionally halogenated aliphatic or aromatic solvents. Indaziflam obtained according to step b) of the preparation process is recrystallized in step c) in a solvent that may be selected from the group consisting of ethanol, methanol, butanol, tert-butanol, propanol, isopropyl alcohol, isoamyl alcohol, phenol, anyone of them alone or mixed with water, dimethylformamide, dimethylacetamide, N-methyl caprolactam, N-ethyl caprolactam, ethyl acetate, n-butyl acetate, hexyl acetate, toluene, benzene, ethylbenzene, xylenes, cumene, cymenes, mesitylene, biphenyl, chlorobenzene, bromobenzene, o-dichlorobenzene, m-dichlorobenzene, p-dichlorobenzene, chloromethane, dichloromethane, trichloromethane, tetrachloromethane, dichloroethane, trichloroethane, trichloroethylene, tetrachloroethylene, pentane, hexane, heptane, or cyclohexane.

The recrystallization in step c) of the preparation process may be achieved by dissolving the indaziflam obtained according to step b) in the selected solvent at any suitable temperature, that may be for example, from room temperature to the reflux temperature of the selected solvent, then indaziflam is caused to precipitate from the solution by any suitable technique, that may be for example, by cooling to any suitable temperature and/or by removing an appropriate part of the selected solvent to cause the precipitation of indaziflam which has a compressibility index lower or equal to 17. Finally, the precipitated indaziflam which has a compressibility index lower or equal to 17 is isolated or recovered from the selected solvent by any suitable technique, that may be for example, by filtration, centrifugation and/or decantation. The person skilled in the art is familiar with the different techniques and steps for the recrystallization of a product.

### Composition Comprising a Crystalline Form of Indaziflam

The present invention also relates to an agrochemical composition which comprises a crystalline form of indaziflam which has a compressibility index lower or equal to 17.

The crystalline form of indaziflam at the agrochemical composition may exhibit an X-ray powder diffractogram (XRD) recorded using Cu-Kα radiation at 25° C. with peaks at diffraction angles 2θ±0.2 degree at 14.3, 16.9, 18.9, 20.1, 22.5 and 25.2 degrees.

The crystalline form of indaziflam at the agrochemical composition may exhibit an X-ray powder diffractogram (XRD) recorded using Cu-Kα radiation at 25° C. with peaks at diffraction angles 2θ±0.2 degree at 14.3, 16.9, 18.9, 20.1, 22.5 and 25.2 degrees, and it may have also additional peaks at diffraction angles 2θ±0.2 degree at 13.0, 16.1, 21.5, 24.6 degrees.

The crystalline form of indaziflam at the agrochemical composition may exhibit an infrared (IR) spectrum with characteristic functional peaks at wavenumbers (±2 cm⁻¹) at 569, 822, 1055, 1422, 1449, 1474, 1527, 1575 cm⁻¹.

The crystalline form of indaziflam at the agrochemical composition may exhibit an infrared (IR) spectrum with characteristic functional peaks at wavenumbers (±2 cm⁻¹) at 569, 822, 1055, 1422, 1449, 1474, 1527, 1575 cm⁻¹, and it may have also additional functional peaks at wavenumbers (±2 cm⁻¹) at 637, 709, 874, 895, 996, 1124, 1306, 1372, 1393, 1638, 2948, 3222, 3319, 3432 cm⁻¹.

The crystalline form of indaziflam at the agrochemical composition may exhibit an X-ray powder diffractogram (XRD) recorded using Cu-Kα radiation at 25° C. with peaks at diffraction angles 2θ±0.2 degree at 14.3, 16.9, 18.9, 20.1, 22.5 and 25.2 degrees, and it may exhibit an infrared (IR) spectrum with characteristic functional peaks at wavenumbers (±2 cm⁻¹) at 569, 822, 1055, 1422, 1449, 1474, 1527, 1575 cm⁻¹. The crystalline form of indaziflam at the agrochemical composition may exhibit an X-ray powder diffractogram (XRD) recorded using Cu-Kα radiation at 25° C. with peaks at diffraction angles 2θ±0.2 degree at 14.3, 16.9, 18.9, 20.1, 22.5 and 25.2 degrees, it may have also additional peaks at diffraction angles 2θ±0.2 degree at 13.0, 16.1, 21.5, 24.6 degrees, it may exhibit an infrared (IR) spectrum with characteristic functional peaks at wavenumbers (±2 cm⁻¹) at 569, 822, 1055, 1422, 1449, 1474, 1527, 1575 cm⁻¹, and it may have also additional functional peaks at wavenumbers (±2 cm⁻¹) at 637, 709, 874, 895, 996, 1124, 1306, 1372, 1393, 1638, 2948, 3222, 3319, 3432 cm⁻¹.

The crystalline form of indaziflam at the agrochemical composition may exhibit a melting point of from 181.4° C. to 183.4° C.

The crystalline form of indaziflam at the agrochemical composition may exhibit a differential scanning calorimetry (DSC) profile having an endothermic melting peak with onset at 182.4° C.±0.2° C. and peak maximum at 182.8° C.±0.2° C.

The crystalline form of indaziflam at the agrochemical composition may exhibit a differential scanning calorimetry (DSC) profile having an endothermic melting peak with onset at 182.4° C.±0.2° C. and peak maximum at 182.8° C.±0.2° C., and it may exhibit an X-ray powder diffractogram (XRD) recorded using Cu-Kα radiation at 25° C. with peaks at diffraction angles 2θ±0.2 degree at 14.3, 16.9, 18.9, 20.1, 22.5 and 25.2 degrees.

The crystalline form of indaziflam at the agrochemical composition may exhibit a differential scanning calorimetry (DSC) profile having an endothermic melting peak with onset at 182.4° C.±0.2° C. and peak maximum at 182.8° C.±0.2° C., it may exhibit an X-ray powder diffractogram (XRD) recorded using Cu-Kα radiation at 25° C. with peaks at diffraction angles 2θ±0.2 degree at 14.3, 16.9, 18.9, 20.1, 22.5 and 25.2 degrees, and the diffractogram may have also additional peaks at diffraction angles 2θ±0.2 degree at 13.0, 16.1, 21.5, 24.6 degrees.

The crystalline form of indaziflam at the agrochemical composition may exhibit a differential scanning calorimetry (DSC) profile having an endothermic melting peak with onset at 182.4° C.±0.2° C. and peak maximum at 182.8° C.±0.2° C., it may exhibit an X-ray powder diffractogram (XRD) recorded using Cu-Kα radiation at 25° C. with peaks at diffraction angles 2θ±0.2 degree at 14.3, 16.9, 18.9, 20.1, 22.5 and 25.2 degrees, and it may exhibit an infrared (IR) spectrum with characteristic functional peaks at wavenumbers (±2 cm⁻¹) at 569, 822, 1055, 1422, 1449, 1474, 1527, 1575 cm⁻¹.

The crystalline form of indaziflam at the agrochemical composition may exhibit a differential scanning calorimetry (DSC) profile having an endothermic melting peak with onset at 182.4° C.±0.2° C. and peak maximum at 182.8° C.±0.2° C., it may exhibit an X-ray powder diffractogram (XRD) recorded using Cu-Kα radiation at 25° C. with peaks at diffraction angles 2θ±0.2 degree at 14.3, 16.9, 18.9, 20.1, 22.5 and 25.2 degrees, the diffractogram may have also additional peaks at diffraction angles 2θ±0.2 degree at 13.0, 16.1, 21.5, 24.6 degrees, it may exhibit an infrared (IR) spectrum with characteristic functional peaks at wavenumbers (±2 cm⁻¹) at 569, 822, 1055, 1422, 1449, 1474, 1527, 1575 cm⁻¹, and it may have also additional functional peaks at wavenumbers (±2 cm⁻¹) at 637, 709, 874, 895, 996, 1124, 1306, 1372, 1393, 1638, 2948, 3222, 3319, 3432 cm⁻¹.

The agrochemical composition which comprises a crystalline form of indaziflam which has a compressibility index lower or equal to 17, may be in the form of a suspension concentrate (SC), granules (GR) in the form of microgranules, spray granules, coated granules and absorption granules, water dispersible granules (WG), water soluble granules (SG), a dispersible concentrate (DC), oil dispersions (OD), wettable powders (WP), water-soluble powders (SP), a water-soluble concentrate (SL), an emulsion concentrate (EC), or a suspoemulsion (SE). The agrochemical composition which comprises a crystalline form of indaziflam which has a compressibility index lower or equal to 17, may be in the form of a suspension concentrate (SC), granules (GR) in the form of microgranules, spray granules, coated granules and absorption granules, water dispersible granules (WG), water soluble granules (SG), a dispersible concentrate (DC) or oil dispersions (OD). The agrochemical composition which comprises a crystalline form of indaziflam which has a compressibility index lower or equal to 17, may be in the form of a suspension concentrate (SC) or water dispersible granules (WG).

The amount of the crystalline form of indaziflam which has a compressibility index lower or equal to 17, in the agrochemical composition may be between 5% and 90% by total weight of the composition. The amount of the crystalline form of indaziflam which has a compressibility index lower or equal to 17, in the agrochemical composition may be between 10% and 80% by total weight of the composition. The amount of the crystalline form of indaziflam which has a compressibility index lower or equal to 17, in the agrochemical composition may be between 15% and 60% by total weight of the composition.

The amount of the crystalline form of indaziflam which has a compressibility index lower or equal to 17, in the agrochemical composition may be between 5% and 90% by total weight of the composition, and the crystalline form of indaziflam may exhibit an X-ray powder diffractogram (XRD) recorded using Cu-Kα radiation at 25° C. with peaks at diffraction angles 2θ±0.2 degree at 14.3, 16.9, 18.9, 20.1, 22.5 and 25.2 degrees. The amount of the crystalline form of indaziflam which has a compressibility index lower or equal to 17, in the agrochemical composition may be between 10% and 80% by total weight of the composition, the crystalline form of indaziflam may exhibit an X-ray powder diffractogram (XRD) recorded using Cu-Kα radiation at 25° C. with peaks at diffraction angles 2θ±0.2 degree at 14.3, 16.9, 18.9, 20.1, 22.5 and 25.2 degrees, and it may exhibit an infrared (IR) spectrum with characteristic functional peaks at wavenumbers (±2 cm⁻¹) at 569, 822, 1055, 1422, 1449, 1474, 1527, 1575 cm⁻¹. The amount of the crystalline form of indaziflam which has a compressibility index lower or equal to 17, in the agrochemical composition may be between 15% and 60% by total weight of the composition, the crystalline form of indaziflam may exhibit an X-ray powder diffractogram (XRD) recorded using Cu-Kα radiation at 25° C. with peaks at diffraction angles 2θ±0.2 degree at 14.3, 16.9, 18.9, 20.1, 22.5 and 25.2 degrees, it may exhibit an infrared (IR) spectrum with characteristic functional peaks at wavenumbers (±2 cm⁻¹) at 569, 822, 1055, 1422, 1449, 1474, 1527, 1575 cm⁻¹, and it may exhibit a differential scanning calorimetry (DSC) profile having an endothermic melting peak with onset at 182.4° C.±0.2° C. and peak maximum at 182.8° C.±0.2° C.

The agrochemical composition which comprises a crystalline form of indaziflam which has a compressibility index lower or equal to 17, may be in the form of a suspension concentrate (SC), granules (GR) in the form of microgranules, spray granules, coated granules and absorption granules, water dispersible granules (WG), water soluble granules (SG), a dispersible concentrate (DC), oil dispersions (OD), wettable powders (WP), water-soluble powders (SP), a water-soluble concentrate (SL), an emulsion concentrate (EC), or a suspoemulsion (SE), and the amount of the crystalline form of indaziflam may be between 5% and 90% by total weight of the composition. The agrochemical composition which comprises a crystalline form of indaziflam which has a compressibility index lower or equal to 17, may be in the form of a suspension concentrate (SC), granules (GR) in the form of microgranules, spray granules, coated granules and absorption granules, water dispersible granules (WG), water soluble granules (SG), a dispersible concentrate (DC) or oil dispersions (OD), and the amount of the crystalline form of indaziflam may be between 10% and 80% by total weight of the composition. The agrochemical composition which comprises a crystalline form of indaziflam which has a compressibility index lower or equal to 17, may be in the form of a suspension concentrate (SC) or water dispersible granules (WG), and the amount of the crystalline form of indaziflam may be between 15% and 60% by total weight of the composition.

The agrochemical composition which comprises a crystalline form of indaziflam which has a compressibility index lower or equal to 17, may further comprise one or more agriculturally acceptable inert additives or adjuvants. The agrochemical composition which comprises a crystalline form of indaziflam which has a compressibility index lower or equal to 17, may further comprise one or more agriculturally acceptable inert additives or adjuvants selected from the group consisting of chelating agents, thickeners, anti-foam agents, pH buffers, antifreeze agents, dispersants, surfactants, emulsifiers, wetting agents or humectants, suspending agents, stabilizers, plant penetrants (or translocators), safeners, spreading agents, compatibility agents, drift retardants, anti-oxidation agents, preservative agents, drift retardants, inverting agents, soil penetrants, UV absorbers, binders, stickers, fertilizers, inert fillers, pigments, colorants, solvents or mixtures thereof.

The agrochemical composition which comprises a crystalline form of indaziflam which has a compressibility index lower or equal to 17, may further comprise one or more herbicide active ingredients. The agrochemical composition which comprises a crystalline form of indaziflam which has a compressibility index lower or equal to 17, may further comprise one or more herbicide active ingredients selected from the group consisting of aclonifen, acrolein, azafenidin, acifluorfen, azimsulfuron, asulam, acetochlor, atrazine, anilofos, amicarbazone, amidosulfuron, amitrole, aminocyclopyrachlor, aminopyralid, amiprofos-methyl, ametryn, alachlor, alloxydim, ioxynil, isouron, isoxachlortole, isoxaflutole, isoxaben, isoproturon, ipfencarbazone, imazaquin, imazapic, imazapyr, imazamethabenz-methyl, imazamox, imazethapyr, imazosulfuron, indanofan, eglinazine-ethyl, esprocarb, ethametsulfuron-methyl, ethalfluralin, ethidimuron, ethoxysulfuron, ethoxyfen-ethyl, ethofumesate, etobenzanid, endothal-disodium, oxadiazon, oxadiargyl, oxaziclomefone, oxasulfuron, oxyfluorfen, oryzalin, orthosulfamuron, orbencarb, oleic acid, cafenstrole, carfentrazone-ethyl, karbutilate, carbetamide, quizalofop-ethyl, quizalofop-P-ethyl, quizalofop-P-tefuryl, quinoclamine, quinclorac, quinmerac, cumyluron, clacyfos, glyphosate, glufosinate, clethodim, clodinafop-propargyl, clopyralid, clomazone, chlomethoxyfen, clomeprop, cloransulam-methyl, chloramben, chloridazon, chlorimuron-ethyl, chlorsulfuron, chlorthal-dimethyl, chlorthiamid, chlorphthalim, chlorflurenol-methyl, chlorpropham, chlorbromuron, chloroxuron, chlorotoluron, ketospiradox, saflufenacil, sarmentine, cyanazine, cyanamide, diuron, diethatyl-ethyl, dicamba, cycloate, cycloxydim, diclosulam, cyclosulfamuron, cyclopyranil, cyclopyrimorate, dichlobenil, diclofop-P-methyl, diclofop-methyl, dichlorprop, dichlorprop-P, diquat, dithiopyr, siduron, dinitramine, cinidon-ethyl, cinosulfuron, dinoseb, dinoterb, cyhalofop-butyl, diphenamid, difenzoquat, diflufenican, diflufenzopyr, simazine, dimethachlor, dimethametryn, dimethenamid, dimethenamid-P, simetryn, dimepiperate, dimefuron, cinmethylin, sulcotrione, sulfentrazone, sulfosate, sulfosulfuron, sulfometuron-methyl, sethoxydim, terbacil, daimuron, thaxtomin A, dalapon, thiazopyr, tiafenacil, thiencarbazone, tiocarbazil, thiobencarb, thidiazimin, thifensulfuron-methyl, desmedipham, desmetryne, tetflupyrolimet, thenylchlor, tebutam, tebuthiuron, tepraloxydim, tefuryltrione, tembotrione, terbuthylazine, terbutryn, terbumeton, topramezone, tralkoxydim, triaziflam, triasulfuron, triafamone, triallate, trietazine, triclopyr, triclopyr-butotyl, trifludimoxazin, tritosulfuron, triflusulfuron-methyl, trifluralin, trifloxysulfuron, tribenuron-methyl, tolpyralate, naptalam, naproanilide, napropamide, napropamide-M, nicosulfuron, neburon, norflurazon, vernolate, paraquat, halauxifen-benzyl, halauxifen-methyl, haloxyfop, haloxyfop-P, haloxyfop-etotyl, halosafen, halosulfuron-methyl, bixlozone, picloram, picolinafen, bicyclopyrone, bispyribac-sodium, pinoxaden, bifenox, piperophos, pyraclonil, pyrasulfotole, pyrazoxyfen, pyrazosulfuron-ethyl, pyrazolynate, bilanafos, pyraflufen-ethyl, pyridafol, pyrithiobac-sodium, pyridate, pyriftalid, pyributicarb, pyribenzoxim, pyrimisulfan, pyriminobac-methyl, pyroxasulfone, pyroxsulam, phenisopham, fenuron, fenoxasulfone, fenoxaprop, fenoxaprop-P, fenquinotrione, fenthiaprop-ethyl, fentrazamide, phenmedipham, butachlor, butafenacil, butamifos, butylate, butenachlor, butralin, butroxydim, flazasulfuron, flamprop, flamprop-M, primisulfuron-methyl, fluazifop-butyl, fluazifop-P-butyl, fluazolate, fluometuron, fluoroglycofen-ethyl, flucarbazone-sodium, fluchloralin, flucetosulfuron, fluthiacet-methyl, flupyrsulfuron-methyl-sodium, flufenacet, flufenpyr-ethyl, flupropanate, flupoxame, flumioxazin, flumiclorac-pentyl, flumetsulam, fluridone, flurtamone, fluroxypyr, flurochloridone, pretilachlor, procarbazone-sodium, prodiamine, prosulfuron, prosulfocarb, propaquizafop, propachlor, propazine, propanil, propyzamide, propisochlor, propyrisulfuron, propham, profluazol, propoxycarbazone-sodium, profoxydim, bromacil, brompyrazon, prometryn, prometon, bromoxynil, bromofenoxim, bromobutide, florasulam, florpyrauxifen, hexazinone, pethoxamid, benazolin, penoxsulam, heptamaloxyloglucan, beflubutamid, beflubutamid-M, pebulate, pelargonic-acid, bencarbazone, pendimethalin, benzfendizone, bensulide, bensulfuron-methyl, benzobicyclon, benzofenap, bentazone, pentanochlor, pentoxazone, benfluralin, benfuresate, fosamine, fomesafen, foramsulfuron, mecoprop, mesosulfuron-methyl, mesotrione, metazachlor, metazosulfuron, methabenzthiazuron, metamitron, metamifop, DSMA (disodium methanearsonate), methiozolin, methyldymuron, metoxuron, metosulam, metsulfuron-methyl, metobromuron, metobenzuron, metolachlor, S-metolachlor, metribuzin, mefenacet, monosulfuron, monolinuron, molinate, iodosulfuron, iodosulfuron-methyl-sodium, iofensulfuron, lactofen, lancotrione, linuron, rimsulfuron, lenacil, 2,2,2-trichloroacetic acid, 2,3,6-trichlorobenzoic acid, 2,4,5-trichlorophenoxyacetic acid, 2,4-D ((2,4-dichlorophenoxyacetic acid), or the agriculturally acceptable salts of these herbicide active ingredients.

### Process for the Preparation of an Agrochemical Composition Comprising a Crystalline Form of Indaziflam

The present invention also relates to a process for the preparation of an agrochemical composition comprising indaziflam, wherein the process comprises a step of pulverizing a powder or a slurry comprising a crystalline form of indaziflam which has a compressibility index lower or equal to 17, and a step of mixing the pulverized powder or slurry with one or more agriculturally acceptable inert additives or adjuvants of the agrochemical composition.

The step of mixing a pulverized powder or slurry comprising a crystalline form of indaziflam which has a compressibility index lower or equal to 17 with one or more agriculturally acceptable inert additives or adjuvants may further comprise one or more herbicide active ingredients.

The agrochemical composition comprising indaziflam in the process for the preparation thereof, may be in the form of a suspension concentrate (SC), granules (GR) in the form of microgranules, spray granules, coated granules and absorption granules, water dispersible granules (WG), water soluble granules (SG), a dispersible concentrate (DC), oil dispersions (OD), wettable powders (WP), water-soluble powders (SP), a water-soluble concentrate (SL), an emulsion concentrate (EC), or a suspoemulsion (SE).

The one or more agriculturally acceptable inert additives or adjuvants in the process for the preparation of an agrochemical composition comprising indaziflam, may be selected from the group consisting of chelating agents, thickeners, anti-foam agents, pH buffers, antifreeze agents, dispersants, surfactants, emulsifiers, wetting agents or humectants, suspending agents, stabilizers, plant penetrants (or translocators), safeners, spreading agents, compatibility agents, drift retardants, anti-oxidation agents, preservative agents, drift retardants, inverting agents, soil penetrants, UV absorbers, binders, stickers, fertilizers, inert fillers, pigments, colorants, solvents or mixtures thereof.

The following examples are included for illustrative purposes only and should not be construed as limitations on the invention claimed herein.

### EXAMPLES

### Materials y Methods

X-ray powder diffractograms (XRD) are recorded in a Panalytical Empyrean powder diffractometer II (Cu K_{α} radiation, λ=1.54178 Å) equipped with an PIXcell3D-Medipix3 detector and operated at V=45 kV, I=40 mA. Regular continues scans are run in a 2θ range of 3.5÷40° with step equal to ˜0.0131°, time/step ˜40 sec, scan speed ˜0.0847°/sec. The powder sample is gently ground and loaded into a standard quartz plate XRD sample holder with 0.5 mm depth and leveled with a piece of glass.

Infrared (IR) measurements are performed using a Thermo Fisher Scientific NICOLET iS5 FT-IR spectrometer and ATR accessory. 16 scans are performed for each measurement with a resolution of 3.813 cm⁻¹. Spectrum region 525-4000 cm⁻¹. A small amount of powder is placed on the diamond crystal, the pressure tower is then lowered down until it pushed firmly against the powder. When the sample is in place, the measurement is collected on the OMNIC software.

DSC measurements are performed using a DSC3 Mettler Toledo equipment. Sample preparation and measurements are performed following the instructions by Mettler Toledo DSC3 manual.

### Example 1: Synthesis of N-[(1R,2S)-2,6-dimethyl-2,3-dihydro-1H-inden-yl]-6-[(1R)-1-fluoroethyl]-1,3-5-triazine-2,4-diamine with monochlorobenzene and N-methylpyrrolidone

This example follows the process of preparation of indaziflam according to step a1), b) and c).

(1R,2S)-2,6-dimethyl-2,3-dihydro-1H-inden-1-amine HCl salt (16.33 Kg, 82.60 mol, 1 equiv.) is dissolved in 81.65 liters of monochlorobenzene and the mixture is heated to 130° C. under a protective gas atmosphere. Next, 1-cyanoguanidine (13.88 Kg, 165.2 mol, 2 equiv.), which is previously dissolved in 49 liters of N-methylpyrrolidone, is added dropwise. Then, the reaction mixture is stirred for 8 h at 130° C. Upon completion, the mixture is cooled to 50° C., and then monochlorobenzene is distilled off under vacuum. Afterwards, the reaction mixture is cooled to room temperature under a protective gas atmosphere and then 32.66 liters of MeOH are added to the flask, followed by dropwise addition of NaOMe 30% solution in MeOH (37.8 Kg, 206.5 mol, 2.5 equiv.) and methyl-(R)-2-fluoropropionate (17.51 kg, 165.2 mol, 2 equiv.). The mixture is stirred for additional 4 h. At the end of the reaction, water is added dropwise and stirred for another 0.5 h. Afterwards, the mixture is filtered, and the cake is washed with water. Finally, the material is dried in oven at 70° C. under vacuum and crystals were collected. Chemical yield obtained is 84%.

### Example 2: Synthesis of N-[(1R,2S)-2,6-dimethyl-2,3-dihydro-1H-inden-yl]-6-[(1R)-1-fluoroethyl]-1,3-5-triazine-2,4-diamine with monochlorobenzene, N-methylpyrrolidone and trimethylsilyl chloride

This example follows the process of preparation of indaziflam according to step a2), b) and c).

To a reactor equipped with a nitrogen inlet and a teflon coated magnetic stir bar, 2.8 liters of monochlorobenzene are charged under nitrogen atmosphere. Trimethylsilyl chloride (TMSCl) (1.05 equiv, 2.60 mol, 329 ml) is added and the reaction mixture is heated to 90° C. A pre-prepared solution of 1-cyanoguanidine (1.1 equiv, 2.73 mol, 229.5 gr) and (1R,2S)-2,6-dimethyl-2,3-dihydro-1H-inden-1-amine (1 equiv, 2.48 mol, 400 gr) in N-methylpyrrolidone (570 ml) is added dropwise over 1.5 hours at that temperature during which a viscous off-white mixture is formed. Once addition of the components is completed, the reaction is mixed for 14 hours. Once termination of the 1st step is visible via HPLC, the reaction is cooled down to room temperature and methyl (R)-2-fluoropropanoate (1.7 equiv, 4.22 mol, 447.6 gr) is added in one portion followed by a dropwise addition of NaOMe/MeOH solution (2.1 equiv, 5.21 mol, 30% w/w, 940 gr). Once termination is visible, the mixture is transferred to a round bottom flask and concentrated under reduced pressure. The resulting solid wax is then recrystallized according to the following procedure: MeOH (5 ml per gr of (1R,2S)-2,6-dimethyl-2,3-dihydro-1H-inden-1-amine) is added to the product and heated until full dissolution is visible. The mixture is stirred at room temperature and water (2.8 ml per gr of (1R,2S)-2,6-dimethyl-2,3-dihydro-1H-inden-1-amine) is added dropwise over 0.5 hr followed by stirring for 2 additional hours. During that time, formation of an off-white precipitate occurs. The resulting suspension is filtered, and the obtained off-white crystals are washed twice with 100 ml of hot water. Finally, the material is dried in an oven at 70° C. under vacuum. The chemical yield obtained is 88%.

### Comparative Example 3: Synthesis of N-[(1R,2S)-2,6-dimethyl-2,3-dihydro-1H-inden-yl]-6-[(1R)-1-fluoroethyl]-1,3-5-triazine-2,4-diamine with 1-methoxy-2-propanol

This comparative example reproduces in a similar way the preparation of N-[(1R,2S)-2,6-dimethyl-2,3-dihydro-1H-inden-yl]-6-[(1R)-1-fluoroethyl]-1,3-5-triazine-2,4-diamine, according to example A8) as instructed in paragraph for compound V-10 of prior art indaziflam preparation process disclosed in EP2231679 B1.

To a reactor 800 ml of 1-methoxy-2-propanol are added, 496 gr of aluminum isopropoxide are added (2 equiv, 2.48 mol). 153.3 gr of cyanoguanidine are then added (1.5 equiv, 1.82 mol). The resulting mixture is then heated to 100° C. for 1 hour. 200 gr (1R,2S)-2,6-dimethyl-2,3-dihydro-1H-inden-1-amine (1 equiv, 1.24 mmol) are added over 30 minutes and mixed for 8 additional hours at 100° C. One termination of reaction is visible the 1-methoxy-2-propanol is distilled off. The reaction is then cooled down to 70° C. and 800 ml of MeOH are added. 88.3 gr of NaOMe (30%) (0.4 equiv, 0.496 mol) and 219.3 gr of methyl-(R)-2-fluoropropionate (1.7 equiv, 2.1 mol) are then added. The reaction is mixed at 70° C. for 8 additional hours until termination is visible via HPLC. The MeOH is distilled off and 1 liter of toluene is added. 1 liter of HCl (16%) are added to the toluene solution and the phases are separated. The aqueous phase is extracted twice with 400 ml toluene and the combined toluene layers are washed with 400 ml of water. The toluene phase is then reduced under pressure and the resulting slurry is dissolved in 1 Liter of MeOH and 400 ml of water are added. The resulting solution is then cooled to 5° C. and NaOH (45%) are added until pH=10 is reached. The resulting slurry is then filtrated and the crystals are washed with hot water and dried under vacuum. The chemical yield obtained is 77%.

### Example 4: Compressibility Index for Crystal Samples from Example 1, Example 2 and Comparative Example 3

Bulk and tapped density measurements are obtained by placing a sample onto a glazed paper. Then, the sample is poured into the tared cylinder until 250 ml mark is reached. The amount of sample is weighed. Rubber bung is fitted into the cylinder. Cylinder is placed in the dropping box and manually tapped (dropped) 50 times with ˜1 second time difference between taps. Compressibility index is calculated from the bulk density and the tapped density as explained in US Pharmacopoeia method <1174> Powder Flow, revision from 20 Jan. 2023.

| | Bulk Density | Tap density | Compressibility |
|---|---|---|---|
| Example | gr/ml | gr/ml | Index |
| Example 1 | 0.56 | 0.65 | 13.6 |
| Example 2 | 0.47 | 0.52 | 10.0 |
| Comparative Example 3 | 0.48 | 0.59 | 19.2 |

Indaziflam crystal samples obtained from Examples 1 and 2 show a compressibility index lower or equal to 17, and therefore a good flowability, as explained in US Pharmacopoeia method <1174> Powder Flow, revision from 20 Jan. 2023. However, the compressibility index for an indaziflam crystal sample obtained from the prior art process in Comparative Example 3 shows a worse value and the flowability of its crystal sample is not good.

1. A crystalline form of indaziflam which has a compressibility index lower or equal to 17.

2. The crystalline form of indaziflam according to claim 1, which exhibits an X-ray powder diffractogram (XRD) recorded using Cu-Kα radiation at 25° C. with peaks at diffraction angles 2θ±0.2 degree at 14.3, 16.9, 18.9, 20.1, 22.5 and 25.2 degrees.

3. The crystalline form of indaziflam according to any of previous claims, which exhibits an infrared (IR) spectrum with characteristic functional peaks at wavenumbers (±2 cm⁻¹) at 569, 822, 1055, 1422, 1449, 1474, 1527, 1575 cm⁻¹.

4. The crystalline form of indaziflam according to any of previous claims, which exhibits a melting point of from 181.4° C. to 183.4° C.

5. The crystalline form of indaziflam according to any of previous claims, which exhibits a differential scanning calorimetry (DSC) profile having an endothermic melting peak with onset at 182.4° C.±0.2° C. and peak maximum at 182.8° C.±0.2° C.

6. A process for the preparation of a crystalline form of indaziflam which has a compressibility index lower or equal to 17, wherein indaziflam is recrystallized in a solvent selected from the group consisting of alcohols, alcohols mixed with water, amides, lactams, esters of carboxylic acids, optionally halogenated aliphatic or aromatic solvents.

7. The process according to claim 6, which comprises:
a1) a first step which is the preparation of (1R,2S)-1-(bisguanidino)-2,6-dimethylindane or an acid addition salt thereof, by reaction of 1-cyanoguanidine with (1R,2S)-2,6-dimethyl-2,3-dihydro-1H-inden-1-amine, in a mixture of a polar aprotic solvent with a dielectric constant higher than 12.0, when measured at 25° C., and a water immiscible solvent;
or alternatively,
a2) a first step which is the preparation of (1R,2S)-1-(bisguanidino)-2,6-dimethylindane or an acid addition salt thereof, by reaction of 1-cyanoguanidine with (1R,2S)-2,6-dimethyl-2,3-dihydro-1H-inden-1-amine, in a mixture which comprises a trimethylsilyl-containing compound, and an optionally alkyl substituted N-alkyl γ-, δ-, ε-lactam or an optionally alkyl substituted γ-, δ-, ε-lactone;
b) a second step which is the addition of a base and methyl (R)-2-fluoropropanoate to (1R,2S)-1-(bisguanidino)-2,6-dimethylindane or an addition salt thereof, obtained in the first step a1) or a2), and
c) recrystallization of indaziflam obtained in step b) in a solvent selected from the group consisting of alcohols, alcohols mixed with water, amides, lactams, esters of carboxylic acids, optionally halogenated aliphatic or aromatic solvents.

8. An agrochemical composition which comprises a crystalline form of indaziflam which has a compressibility index lower or equal to 17.

9. The agrochemical composition according to claim 8, wherein the composition is in the form of a suspension concentrate (SC), granules (GR) in the form of microgranules, spray granules, coated granules and absorption granules, water dispersible granules (WG), water soluble granules (SG), a dispersible concentrate (DC), oil dispersions (OD), wettable powders (WP), water-soluble powders (SP), a water-soluble concentrate (SL), an emulsion concentrate (EC), or a suspoemulsion (SE).

10. The agrochemical composition according to any of claims 8 to 9, wherein the amount of the crystalline form of indaziflam in the composition is between 5% and 90% by total weight of the composition.

11. The agrochemical composition according to any of claims 8 to 10, wherein it further comprises one or more agriculturally acceptable inert additives or adjuvants.

12. The agrochemical composition according to claim 11, wherein the one or more agriculturally acceptable inert additives or adjuvants are selected from the group consisting of chelating agents, thickeners, anti-foam agents, pH buffers, antifreeze agents, dispersants, surfactants, emulsifiers, wetting agents or humectants, suspending agents, stabilizers, plant penetrants (or translocators), safeners, spreading agents, compatibility agents, drift retardants, anti-oxidation agents, preservative agents, drift retardants, inverting agents, soil penetrants, UV absorbers, binders, stickers, fertilizers, inert fillers, pigments, colorants, solvents or mixtures thereof.

13. The agrochemical composition according to any of claims 8 to 12, wherein it further comprises one or more herbicide active ingredients.

14. A process for the preparation of an agrochemical composition comprising indaziflam, wherein the process comprises a step of pulverizing a powder or a slurry comprising a crystalline form of indaziflam which has a compressibility index lower or equal to 17, and a step of mixing the pulverized powder or slurry with one or more agriculturaly acceptable inert additives or adjuvants of the agrochemical composition.

15. The process for the preparation of an agrochemical composition comprising indaziflam according to claim 14, wherein the crystalline form of indaziflam and the one or more agriculturally acceptable inert additives or adjuvants are mixed with one or more herbicide active ingredients.
